(19) United States
(12) Patent Application Publication
(10) Pub. No.: US 2003/0074075 A1
(43) Pub. Date: Apr. 17, 2003

(54) Expandable implant for partial disc replacement and reinforcement of a disc partially removed in a discectomy and for reduction and maintenance of alignment of cancellous bone fractures and methods and apparatuses for same

(76) Inventors: James C. Thomas JR., Las Vegas, NV (US); David C. Forster JR., Menlo Park, CA (US); Gregory M. Mast, Fremont, CA (US); Travis Rowe, Fremont, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/229,949

(22) Filed: Aug. 27, 2002

Related U.S. Application Data
(60) Provisional application No. 60/315,268, filed on Aug. 27, 2001.

(51) Int. Cl. A61F 2/44; A61F 2/28; A61B 17/68

(52) U.S. Cl. 623/17.16; 623/908; 606/72; 623/23.53

(57) ABSTRACT

Expandable implants for intervertebral disc repair, and methods and apparatuses for delivering the same into the disc. The present implants can also be used for repair of bone fractures. The implants generally comprise a compressed form having a size adapted for insertion into a defect in the intervertebral disc, and a composition that allows the implant to expand from the compressed form into an expanded form after the implant is inserted into the defect. The expanded form of the implant has a configuration that fills the defect in the disc. The defect in the disc can be an annular defect that resulted from repair of a herniation of the disc, or a nucleus that needs to be repaired. The composition used to make the implant can comprise a shape memory alloy (SMA) or any other suitable material.

### CROSS-REFERENCE TO RELATED APPLICATION

This application claims the benefit of U.S. Provisional Application No. 60/315,268 filed on Aug. 27, 2001.

### FIELD OF THE INVENTION

The present invention relates to expandable implants for partial disc replacement and repair of cancellous bone fractures, and more specifically, to expandable implants and methods for delivering the same that can be used to repair an annular and nuclear defects in a disc, as well as repairing various types of cancellous bone fractures.

### BACKGROUND OF THE INVENTION

A lumbar intervertebral disc comprises a mechanical and flexible component to the spine to allow better support of the vertebral body and the spinal column. The disc is made of two components, an annulus and a nucleus. The annulus is the outer structure and is composed of multiple layers of collagen fibers. Each fiber is uniquely oriented at 30 degrees to the adjacent fiber. When intact the annulus can support pressures of up to 100-120 lbs per square inch. The nucleus is the inner structure and is composed of a different collagen, which is largely water and in a gelatinous form. The nucleus is held under pressure in the center of the intact disc by the intact annulus. (See FIGS. 1a & 1b). Unfortunately, the annulus is prone to tears and traumatic events. When a tear occurs from the periphery of the annulus to the center of the nucleus, this comprises a radial annular tear. This will allow the nucleus to rupture through the annular tear into and towards the spinal canal (see FIGS. 2a & 2b). This ruptured nucleus material puts pressure on the neural and ligamentous structures causing back pain and often pain down the posterior aspect of the buttock and leg. This particular symptom is named sciatica.

Conservative treatment is often performed. However, when conservative treatment fails and pain is intractable or neurologic deficit exists, surgery is performed. In this particular surgery, a small opening (a laminotomy) is made in the back of the spinal bone structure to allow access to the spinal canal. The nerve root and thecal sac are gently retracted and the hernia identified. The hernia is essentially removed with micro surgical tools and instruments. A defect is left in the annulus. Nothing is placed in the annular defect. (See FIGS. 3a & 3b). The surgeon depends upon a fibroblastic response to repair the defect with scar tissue.

However, the vascularity of the adult intervertebral disc is poor. The disc is the largest avascular structure in the human body next to the cornea of the eye. As a result, healing with scar tissue is very fragile, if it occurs at all, and often, over a period of years, further degeneration of the annular and nuclear structures occurs. The disc space narrows as a result of this progressive degenerative phenomena and this causes new problems such as root compression in the exit zone of the spinal canal. This area is known as the foramen. This may result in the patient having increased or recurrent symptoms, and a subsequent surgical fusion may be required for the patient. The statistics vary for the number of patients who have laminectomy and discectomy and subsequently require fusion. They may be as high as 70% over a ten year period.

In addition to the problems that exist with the repair of annular defects, the same obstacles have been present with respect to nuclear defects. Because the nucleus often ruptures through tears in the annulus, there often is an inadequate amount of residual nucleus for the disc to provide its weight bearing support and compression functions. As a result, there exists a need for an implant that can be inserted into the nucleus to simulate the function and structure of the original nucleus.

Furthermore, conditions similar to those present in a damaged disc exist in other parts of the human body. Particularly, areas where cancellous bone fractures occur have been difficult to adequately repair. For example, areas such as the distal radius and the plateau of the tibia adjacent to the knee often suffer cancellous fractures and result in further complications such as a collapse and alteration of alignment of joints. Also, fractures in areas such as the thoracic or lumbar spine are common, particularly in elderly patients who suffer from weak osteoporotic bones. Known treatments for many of these types of fractures have been largely inadequate. For example, some treatments have included injection of liquid bone cement (vertebroplasty) into the fracture, insertion of a prosthetic balloon (kyphoplasty) that is inflated to create a cavity where cement can be subsequently injected. Overall, the known techniques have been inadequate to reliably fill the void of the fracture, and at the same time reinforce the fracture and support its realignment/reduction.

Accordingly, there exists a need for devices and methods for treating damaged discs and bone fractures that overcome the problems and inadequacies of treatments currently available. Particularly, there is a need for expandable implants that effectively repair annular defects, nuclear defects, and cancellous bone fractures.

### SUMMARY OF THE INVENTION

The present invention relates to expandable implants for intervertebral disc repair, and methods and apparatuses for delivering the same into the disc. The present implants can also be used for repair of bone fractures. The implants generally comprise a compressed form having a size adapted for insertion into a defect in the intervertebral disc, and a composition that allows the implant to expand from the compressed form into an expanded form after the implant is inserted into the defect. The expanded form of the implant has a configuration that fills the defect in the disc. The defect in the disc can be an annular defect that resulted from repair of a herniation of the disc, or a nucleus that needs to be repaired. The composition used to make the implant can comprise a shape memory alloy (SMA) or any other suitable material.

When the implant is made from an SMA, the compressed form is a non-memory shape that is retained until the implant is activated by temperature or electrical current, such that activation transforms the expandable implant to a predetermined memory shape that defines the expanded form.

Various devices can be used to insert the present implants into the area being treated. The devices are adapted to retain the implant while the device is inserted into the intervertebral disc, and to controllably release the implant therein.

### BRIEF DESCRIPTION OF THE DRAWINGS

FIG. 1a shows an axial view of a normal disc and the spinal cord;

FIG. 1b shows a side view of a normal disc and the spinal cord;

FIG. 2a shows an axial view of a ruptured disc putting pressure on the spinal cord;

FIG. 2b shows a side view of a ruptured disc putting pressure on the spinal cord;

FIG. 3a shows an axial view of the ruptured disc of FIG. 2a after the herniation has been removed and an annular defect remains;

FIG. 3b shows a side view of the ruptured disc of FIG. 2b after the herniation has been removed and an annular defect remains;

FIG. 4a shows an implant for treatment of an annular defect, the implant having a “figure eight” configuration;

FIG. 4b shows an implant for treatment of an annular defect, the implant having a “mushroom” shape configuration;

FIG. 4c shows an implant for treatment of an annular defect, the implant having a “brillopad” wiry shape;

FIG. 5 shows a template that can be used to measure an annular defect and simulate various implants;

FIG. 6a shows a disc after a hernia has been removed and the annular defect is empty;

FIG. 6b shows an implant in its unexpanded form prior to insertion into the annular defect;

FIG. 6c shows the implant of FIG. 6b inserted into the annular defect of FIG. 6a, wherein the implant is in its expanded form;

FIG. 7 shows a forcep-like device for inserting an implant into an annular defect;

FIG. 8a shows an implant having a stent basket construction, wherein the implant is disposed over an insertion device;

FIG. 8b shows the stent basket implant fastened to the insertion device;

FIG. 9 shows a closer view of the stent basket implant of FIGS. 8a and 8b;

FIG. 10 shows a pair of barbs extending from the body of the stent basket implant;

FIG. 11a shows an insertion rod device for delivery of a stent basket implant into an annular defect;

FIG. 11b shows loading the stent basket onto the insertion rod device;

FIG. 11c shows additional steps for loading the stent basket onto the insertion rod device;

FIG. 12 shows the delivery of the stent basket implant into the annular defect;

FIG. 13 shows the delivery and release of the stent basket implant into the annular defect;

FIG. 14 shows another implant for treatment of an annular defect, wherein the implant is a stent basket;

FIG. 15 shows another implant for treatment of an annular defect, wherein the implant is a modified stent basket;

FIG. 16 shows another implant for treatment of an annular defect, wherein the implant is a stent plug;

FIG. 17 shows another implant for treatment of an annular defect, wherein the implant is a winged plug;

FIG. 18 shows another implant for treatment of an annular defect, wherein the implant is an inflatable plug;

FIG. 19 shows another implant for treatment of an annular defect, wherein the implant is a spider staple;

FIG. 20 shows another implant for treatment of an annular defect, wherein the implant is a ratchet plug;

FIG. 21 shows another implant for treatment of an annular defect, wherein the implant is a goblet plug;

FIG. 22 shows another implant for treatment of an annular defect, wherein the implant is a goblet device;

FIG. 23 shows another implant for treatment of an annular defect, wherein the implant is a goblet wire device;

FIG. 24 shows another implant for treatment of an annular defect, wherein the implant is a tubular plug;

FIG. 25 shows another implant for treatment of an annular defect, wherein the implant is a modified tubular plug

FIG. 26 shows another implant for treatment of an annular defect, wherein the implant is a spring barb;

FIG. 27a shows an implant for repair of a nucleus, wherein the implant is wires packed into the nucleus to form a spring pad;

FIG. 27b shows an implant for repair of a nucleus, wherein the implant is delivered into a flexible bag that was inserted into the nucleus;

FIG. 28 show a delivery gun for insertion and delivery of an implant for treatment of a nucleus;

FIG. 29a shows a needle for use with a delivery gun for inserting and delivering an implant for treatment of a nucleus;

FIG. 29b shows the needle of FIG. 29a for use with a delivery gun for inserting and delivering an implant for treatment of a nucleus;

FIG. 29c shows a needle having a side port for use with a delivery gun for inserting and delivering an implant for treatment of a nucleus;

FIG. 29d shows the needle of FIG. 29c for use with a delivery gun for inserting and delivering an implant for treatment of a nucleus;

FIG. 30a shows a delivery gun for insertion and delivery of an implant for treatment of a nucleus, wherein a replaceable cartridge and a body are not adjoined;

FIG. 30b shows the delivery gun of FIG. 30a, wherein the replaceable cartridge and the body are adjoined; and

FIG. 31 shows an implant for repair of a nucleus, wherein the implant is microcellular spheres.

### DETAILED DESCRIPTION

The expandable implants of the present invention are suitable for several applications, particularly annular and/or nuclear defects in damaged discs and a wide range of bone fractures. Several possible configurations can be made from a number of different materials.

Overview of Suitable Materials

The present implants are preferably elastic and susceptible to withstanding long term implantation into a mammalian body. Examples of suitable materials include shape memory alloys (SMAs), superelastic SMAs, nitinol, MP35, Elgiloy, spring steel, and any plastic elastic material or other material suitable for such implantation. For simplicity and clarity, many of the embodiments described herein are discussed as being made from a SMA, particularly nitinol, but it is understood that the benefits and features of the present invention are not limited to an SMA or nitinol, and can be achieved by using any of other suitable materials.

SMAs are materials that have the ability to return to a predetermined shape. The return is the result of a change of phase or structure that can be triggered by an external stimulus such as temperature change or electrical current. For example, when one type of SMA is below transformation temperature, it has a low yield strength and can be deformed into a new shape that it will retain while it is below its transformation temperature. However, when the material is heated above its transformation temperature, it undergoes a change in crystal structure that causes it to return to its original shape. If the SMA encounters any resistance during this transformation, it can generate extremely large forces. Thus, SMAs provide a good mechanism for remote actuation. One preferred shape memory material is an alloy of nickel and titanium called nitinol. Nitinol has desirable electrical and mechanical properties, a long fatigue life, high corrosion resistance, and has similar properties to residual annular tissue and cartilaginous tissues. Other SMAs can comprise, for example, alloys of copper, zinc and aluminum or copper, aluminum and nickel. For the present invention, SMA materials or a hybrid with SMA materials can be used to make implants to reconstruct the annular and/or nuclear defects after human discectomy surgery, as well as a variety of bone fractures experienced throughout the human body.

Another type of shape memory alloys are called superelastic SMAs, which can be compressed into a small shape and upon release automatically expand to a predetermined shape. Thus, no external activation, such as temperature or electrical stimulation, is required. One preferred superelastic SMA is superelastic nitinol, which has similar properties to the SMA nitinol discussed above, but because it is a superelastic SMA does not require activation. The superelastic nitinol, or other suitable superelastic SMA, can be compressed into a small package, placed into a surgical deficit such as an annular or nuclear defect or bone fracture and, upon release, expand to a predetermined shape to fill the deficit.

Treatment of Annular Defects

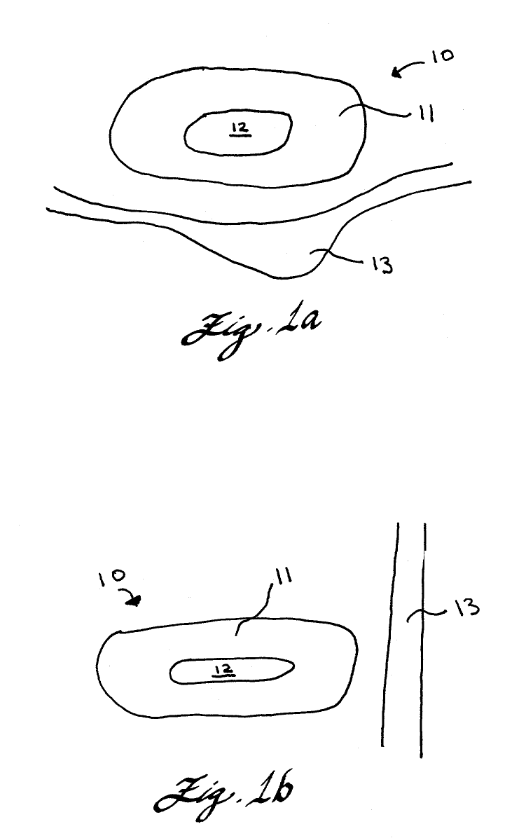

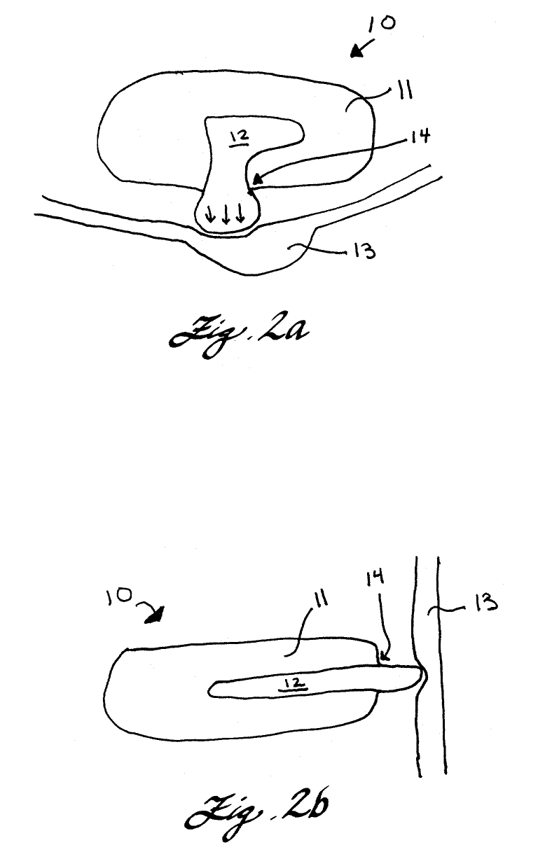

The implants of the present invention are advantageous for treatment of annular defects. The implants can be made from materials such as nitinol and are inserted into the annular defect to reinforce the annulus and restore elasticity to the disc. FIGS. 1 to 3 illustrate a normal disc, a ruptured disc, and a disc that has undergone a discectomy.

Referring to FIG. 1a, an axial view of a normal, unruptured disc 10 is shown. The disc 10 comprises an annulus 11 surrounding a nucleus 12. The spinal cord or nerve 13 is shown in close proximity to the disc, but no portion of the disc is putting pressure on the nerve. FIG. 1b shows a side view of the disc 10 of FIG. 1a.

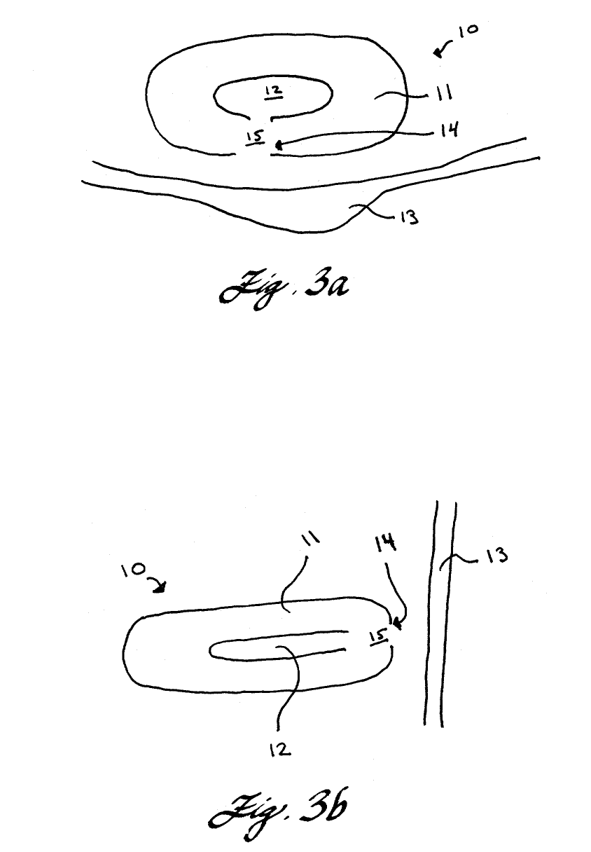

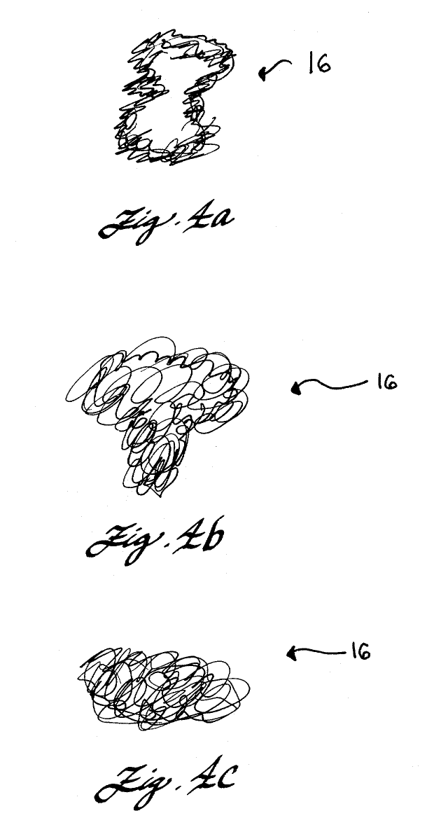

Referring to FIG. 2a, an axial view of a ruptured, herniated disc 10 is shown. The annulus 11 has suffered an annular tear 14, which allowed a portion of the nucleus 12 to rupture through the annulus and put pressure on the nerve 13 (i.e. sciatica) FIG. 2b shows a side view of the ruptured disc 10 of FIG. 2a.

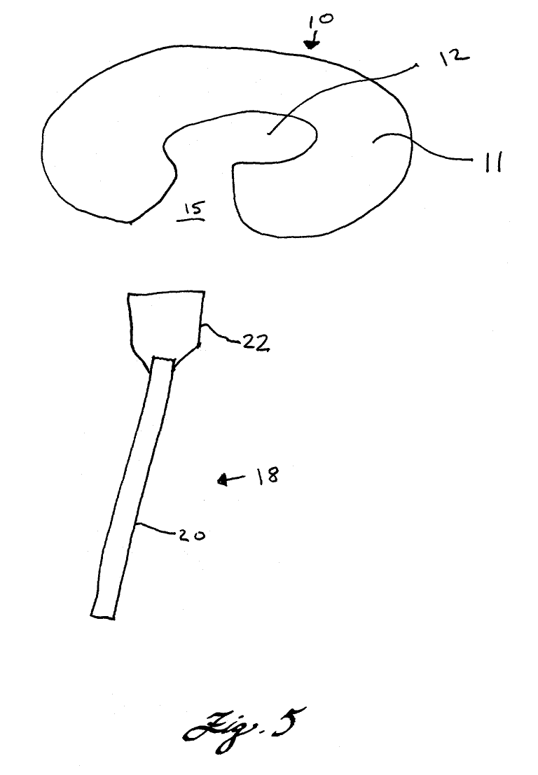

Referring to FIG. 3a, an axial view is shown of the disc 10 after a partial discectomy has been performed to remove the hernia. After the hernia has been removed, the annular tear 14 is still present, but rather than having the portion of the nucleus ruptured through the annulus 11, there remains an annular defect 15, which in effect is an empty space. As noted above, the common practice is to leave the annular defect 15 empty, and rely on fibroblastic growth and scar tissue to fill the defect. FIG. 3b shows a side view of the disc 10 of FIG. 3a.

The implants of the present invention are used to repair the annular defect 15 by filling in the empty space, which provides strength and elasticity to the damaged portion of the annulus and prevents additional portions of the nucleus from exiting the disc. As will become evident, a wide variety of implants can be used to repair the annular defect.

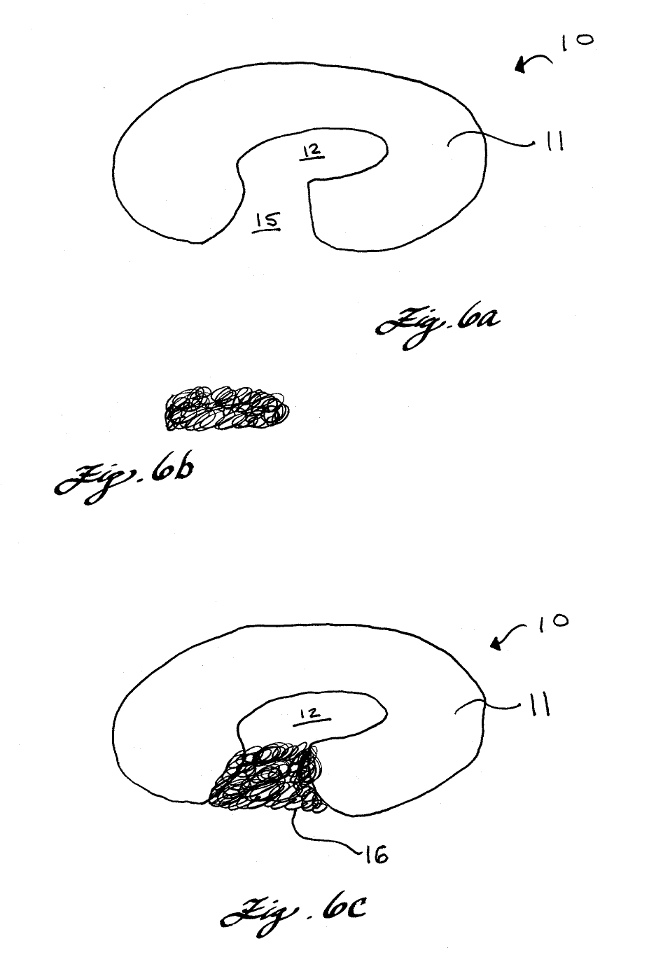

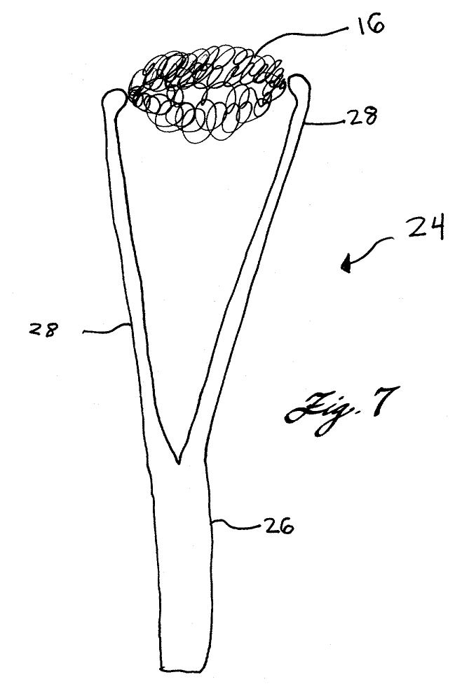

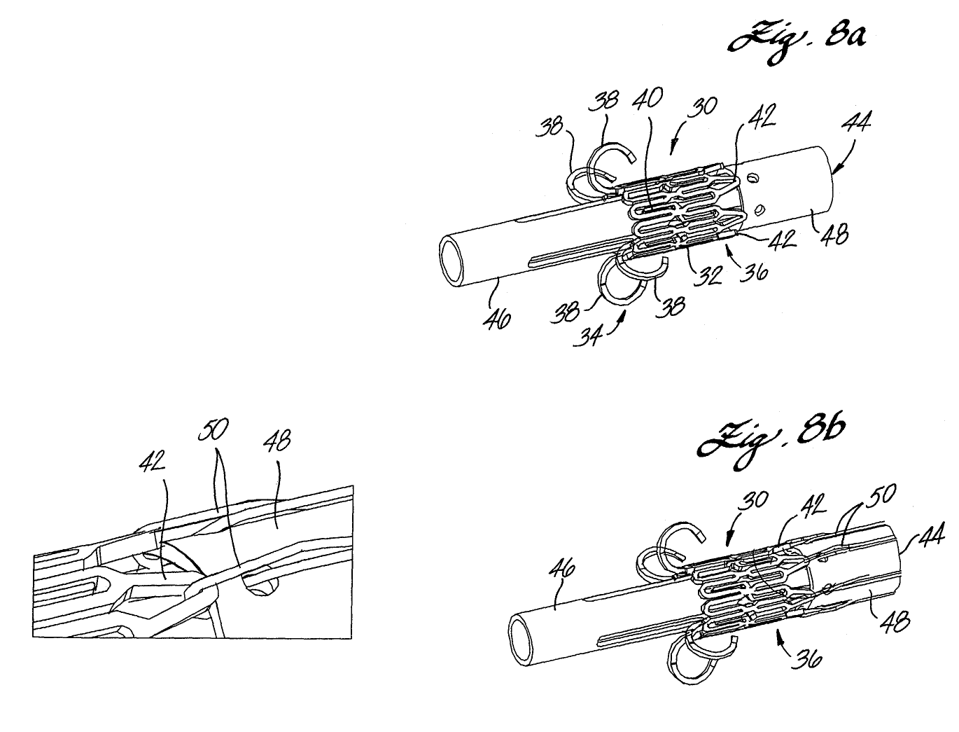

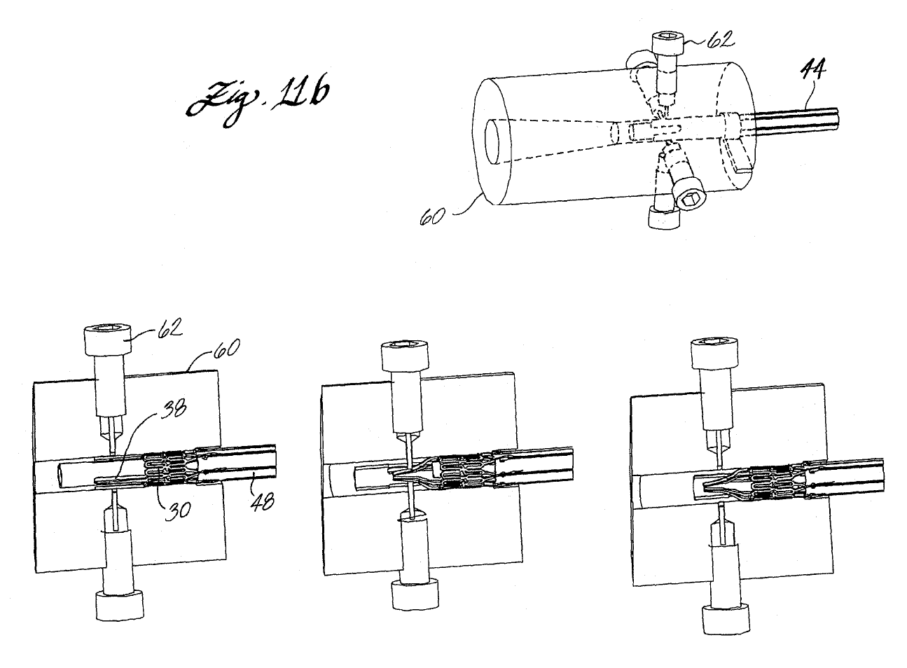

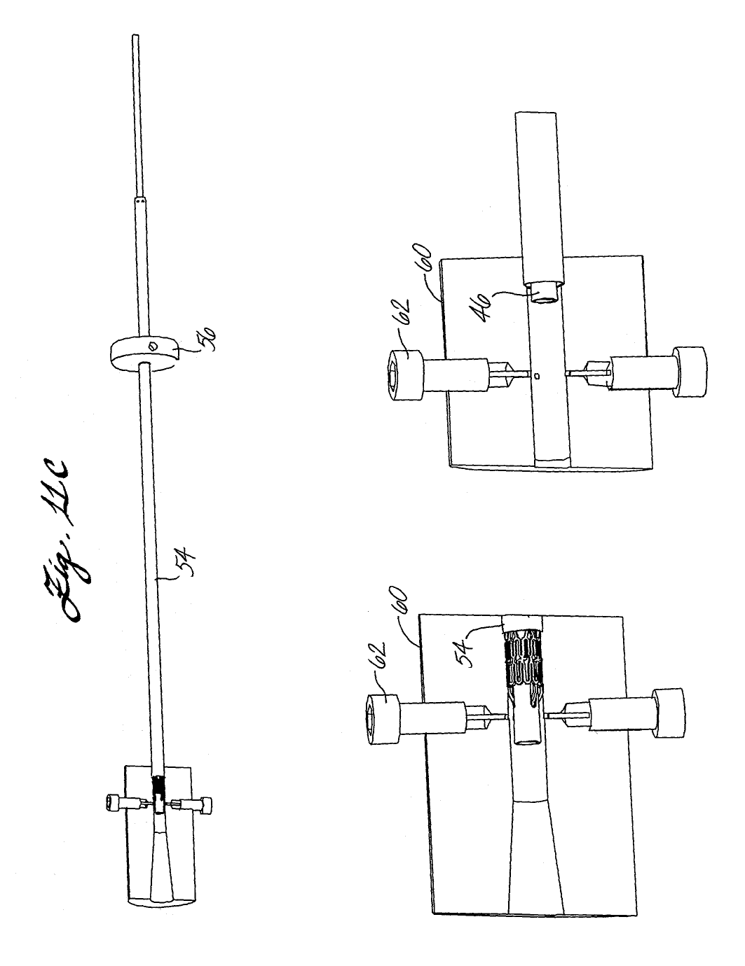

With respect to nitinol implants, the fibers may be oriented at about 30 degrees to each other to simulate the annular structure and anatomy of human discs. While a 30 degree orientation for nitinol fibers is favorable for simulating annular anatomy, it is understood that other orientation angles can be used to provide sufficient tear strength. Because defects in the annulus vary depending on the extent of disc herniation and surgical resection, the structure of the implant used can be varied and customized. In addition to varying the orientation of fibers woven together, the implants can include a wide range of combinations of textures, solid/semi-solid constructions, and porous surfaces. Furthermore, the implants can be configured to any necessary shape, such as a wedge, square, circle, rectangle, cone, cylinder, or any combination therefor. FIGS. 4a to 4c show a few sample combination shapes of an implant 16 of this invention, including a “FIG. 8” configuration (FIG. 4a), a “mushroom” shape (FIG. 4b), and a “brillopad” wiry shape (FIG. 4c). Each of the implants 16 would be designed to fill the specific annular defects 15 present in the disc 10, including corresponding to the curvilinear diameter of the annulus.

After a surgical discectomy is performed, the annular defect 15 can be measured with a small template designed to simulate various implants. The template is an optional device that can be used to measure the size of the annular defect to choose the implant. Referring to FIG. 5, a template 18 can generally comprise a handle 20 with a template head 22. The template head 22 can be any an shape and size, and is designed to insert into the annular defect to determine the appropriate size and shape of the implant 16. The template head can be either permanently or removably adjoined to the handle.

When the implant is made from an SMA such as nitinol, the implant is activated by temperature change or electrical current to cause the implant to expand to its memory shape. For instance, at room temperature the implant may be in its martensite form (more deformable, lower temperature phase). However, when the nitinol implant is inserted into position, the temperature of the body will naturally heat up the nitinol causing it to transform to its austenite form (more rigid, higher temperature phase). The nitinol implant will expand to fill the defect and reinforce the damaged annulus. Based on the various percentages of materials in the implant, the transformation temperature of the implant can be predetermined. The transformation temperature should be high enough so that the implant will remain in the martensite form outside of the body and will not be reduced to its martensite form by the body temperature surrounding the implant after insertion. In the case of the implant being made from a superelastic SMA, activation is not necessary and expansion occurs upon the release of the material to the new area.

The implants can also have adjustable percentages of enlargement depending on the size of the defect. Degree of enlargement can be adjusted by selection of a particular alloy combination or ratio. For example, excess nickel (up to 1%) strongly depresses the transformation temperature and increases the yield strength of the austenite form. Also, iron and chromium can be used to lower the transformation temperature, and copper can be used to decrease hysteresis and lower the deformation stress of the martensite form.

The implants used for treatment of annular defects reinforce the damaged corner of the disc and the annulus. It also acts as a scaffold to promote fibrous ingrowth, by allowing the structure of scar tissue to occur on a more sophisticated basis. It also reduces the asymmetrical collapse that can occur because of the resection of the disc on the posterior longitudinal corner that results from the trauma of injury and/or surgery. Herniations more often than not occur on the left or right side, because the posterior longitudinal ligament reinforces the central portion of the disc. The implant may serve to reduce the degenerative phenomena common to discectomy treatment and potentially reduce the number of patients requiring secondary fusion surgery. By immediately strengthening the annular defect, improved post operative recovery may result as well.

The implants can be designed to expand into the fibrous tissue of the annulus and up to the edge of the nucleus, or slightly into the nucleus, and lodge themselves successfully into the residual disc tissue. Residual disc tissue is present because the surgeon only removes, in general, the portion of the disc that is protruding or ruptured. Generally, anywhere from 50-80% of the residual disc tissue is still present after surgery. This ability to lodge upon expansion into the residual disc tissue prevents the device from being displaced by normal post-operative activities, such as standing, walking, bending or twisting. It is not intended to act as a fusion device and, therefore, does not result in bone growth. On the other hand, the device is designed to promote fibrous tissue ingrowth and reinforces the weakened area of the annulus with its mechanical structure.

Modifications such as placing a collagen type coating or a bio-material onto or into the device to promote annular reconstruction and fibroblastic ingrowth can also be appropriate. A carrier for autologous chondrocyte cells can also be provided to promote regrowth of disc tissue and aid in the repair of the disc. Synthetics that are known to be biocompatible, such as Gortex™ or Teflon™, or other materials, can be applied or interwoven into the nitinol implant to reduce or prevent contact of the implant with neurologic tissue (present on the posterior aspect of the implant) or on the inner circumference of the implant adjacent to the nucleus.

As is apparent from the discussion above, the implants 16 of the present invention can vary widely depending on the particular application. To further illustrate the structural aspects of the implants, example embodiments will be discussed in greater detail. These embodiments are only illustrative of the inventive concepts and are not intended to limit the scope of the claims recited herein.

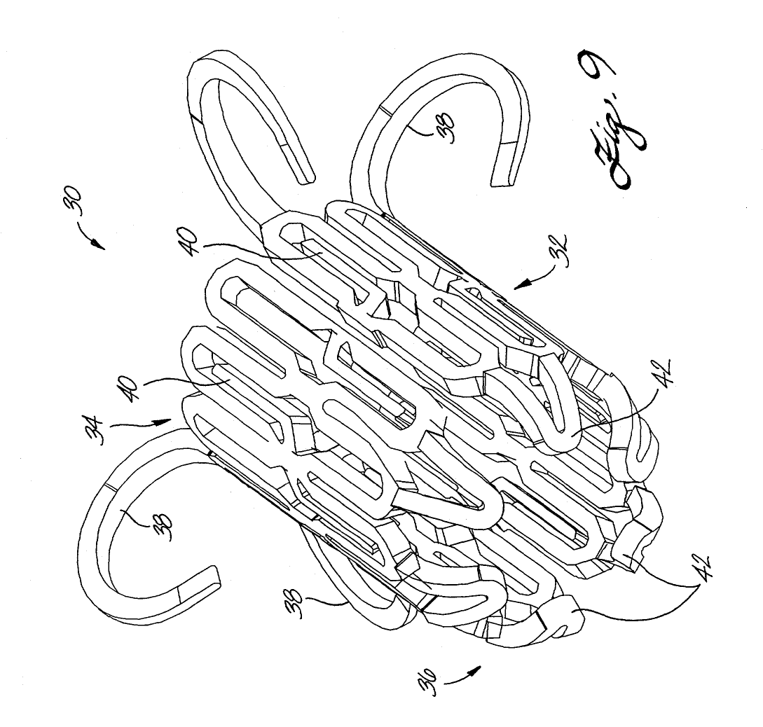

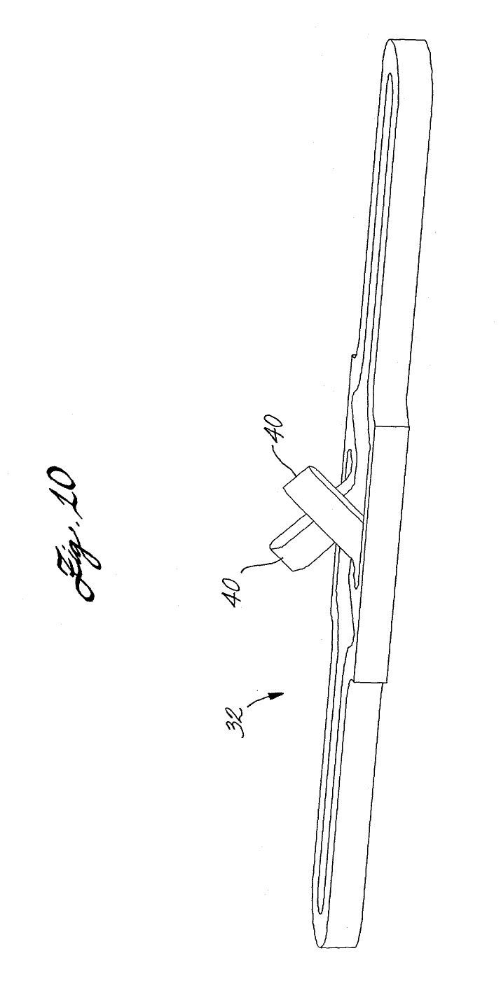

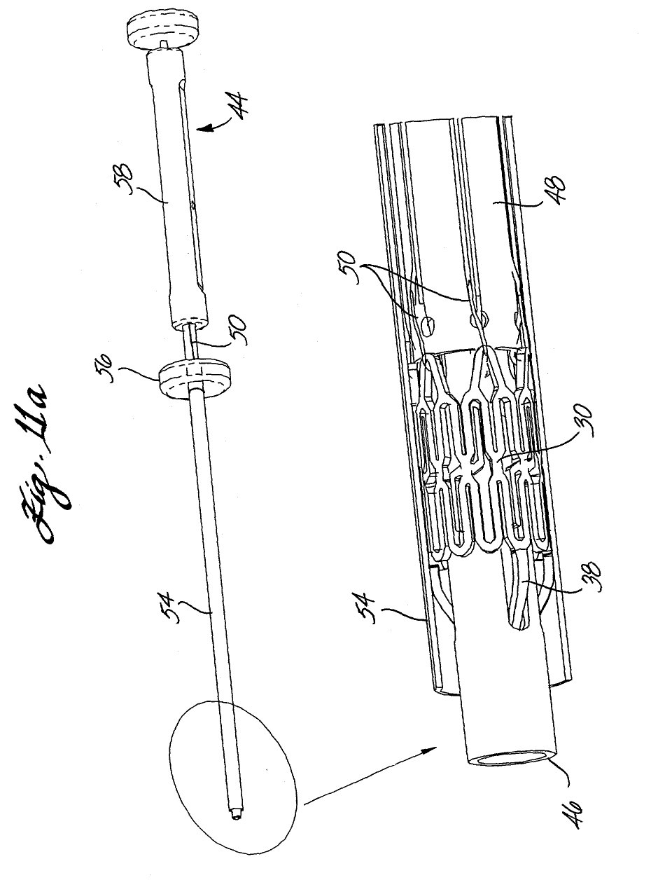

Referring to FIGS. 6a to 6c, the ruptured disc 10 is shown before and after insertion of the implant 16. More specifically, FIG. 6a shows the disc 10 after the hernia has been removed and with the annular defect 15 empty. FIG. 6b shows the implant 16 in its unexpanded form prior to insertion into the annular defect. FIG. 6c shows the annular defect 15 with the implant 16 inserted therein, and the implant 16 fully expanded to its memory form. The implant 16 prevents the residual nucleus 12 from further rupture through the annulus 11. It is understood that the implant 16 could be an SMA, a superelastic SMA, or any other suitable material, that changes from an unexpanded to expanded form either automatically upon release into the annular defect or by some form of activation.

The implant can be inserted into the annular defect by a wide range of implantation devices that are suitable for grasping the implant 16 and precisely positioning the implant within the annular defect. FIG. 7 shows a basic, forcep-like implantation device 24 comprising a body 26 having a pair of arms 28 extending outward. The arms are movable with respect to the body, which allows the surgeon to directly control release of the implant.

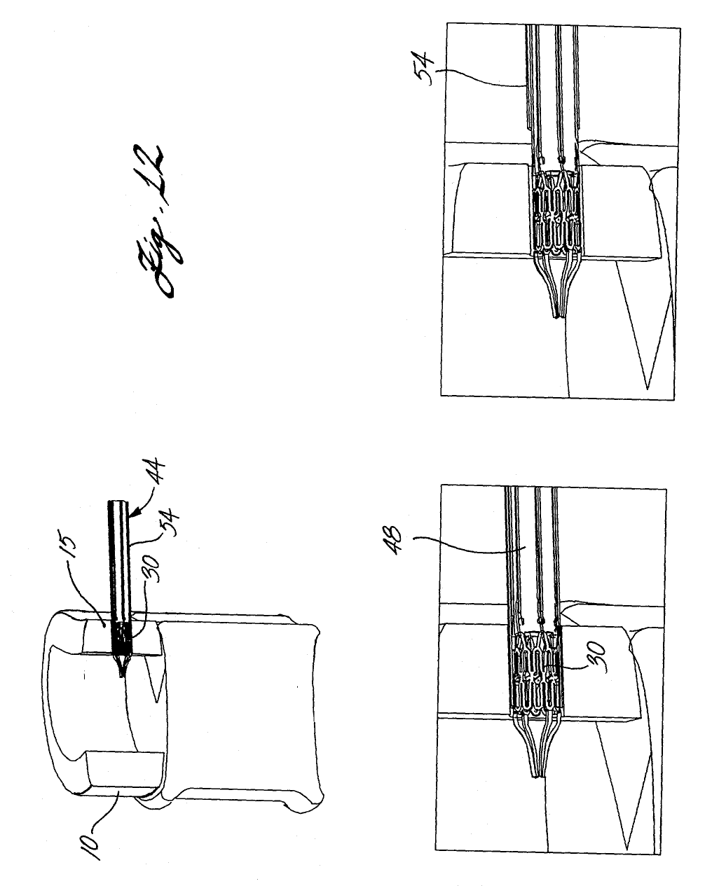

FIGS. 8a, 8b, and 9 show another embodiment of the present implant for treatment of annular defects. Here, the implant is a stent basket 30. The stent basket 30 in FIG. 8a is shown disposed over an insertion rod that is used to insert the stent basket into the annular defect. The stent basket 30 generally comprises a body 32, having a distal end 34 and a proximal end 36 opposite the distal end. The distal end 34 further comprises four expandable retention legs 38. The retention legs 38 are designed to engage the annulus along the portion of the annulus defining the annular defect, such that the stent basket is fixedly engaged within the annular defect. Body 32 has a generally cylindrical shape and is hollow between the distal end and proximal end. This construction allows the body 32 to be radially compressed prior to insertion into the annular defect, and then be radially expanded after insertion. The body is shown having a non-solid exterior surface, such that radial expansion of the body allows portions of the body to extend outward. More specifically, the body 32 comprises a plurality of barbs 40 that help secure the stent basket to the annulus.

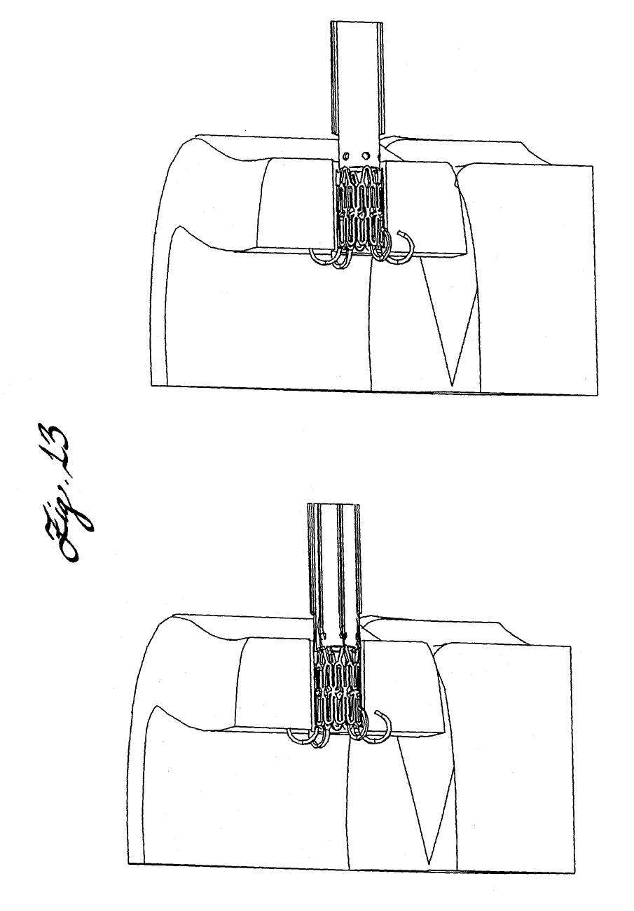

Referring to FIG. 9, the stent basket 30 is shown with the retention legs 38 substantially expanded, while the body 32 is not fully radially expanded. When the body 32 is not fully expanded, the barbs 40 are in uniform orientation with the rest of the body such that a relatively smooth surface is defined by the body. FIG. 10 shows a close-up view of a portion of the stent body 32 after the body has radially expanded. In this expanded form, the barbs 40 extend outward from the body at specified angles, such that the barbs 40 can penetrate part way into the annulus to secure the stent basket and prevent the stent basket from entering or exiting the annular defect. The barbs shown in FIG. 10 are oriented in opposite directions to one another to provide a more secure engagement with the annulus and prevent posterior and anterior migration. The stent basket 30 further comprises a plurality of retention arms 42 at the proximal end 36. The retention arms 42 are designed to be engaged by the insertion device that is used to insert the implant into the annular defect.

The stent basket 30 is preferably made of nitinol or superelastic nitinol. As with the implants 16 discussed above, however, the stent basket 30 can be made from any other suitable material. The structure of the stent basket in its unexpanded and expanded forms is more fully shown by the delivery system/method used to insert the stent basket into the annular defect.

The delivery and insertion of the stent basket is preferably carried out by a multi-component insertion rod device. Referring to FIGS. 8a and 8b, a portion of an insertion rod device 44 is shown, wherein the stent basket 30 is positioned thereon. More specifically, the stent basket is positioned on an inner rod portion 46 of the insertion rod device 44. The insertion rod device 44 further comprises a holding sleeve 48, which is positioned adjacent the proximal end 36 of the stent basket. The holding sleeve 48 is designed for engaging the retention arms 42 of the stent basket by being fastened to the retention arms by a suture material 50. FIG. 8b shows the holding sleeve 48 adjoined to the fastening arms 42 by the suture material 50. FIGS. 8a and 8b illustrate the first two steps of preparing the stent basket 30 for delivery into the annular defect, namely placing the stent basket over the inner rod portion 46 and threading the suture material 50 to fasten the holding sleeve 50 to the retention arms 42.

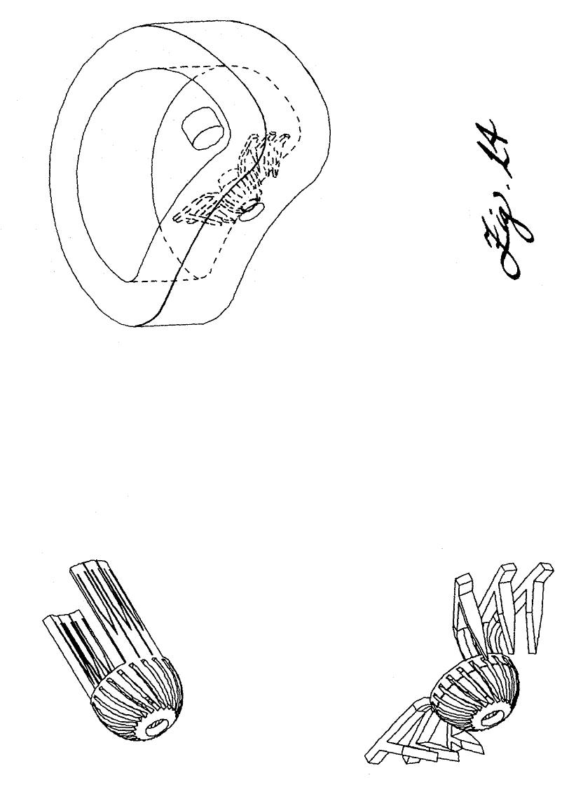

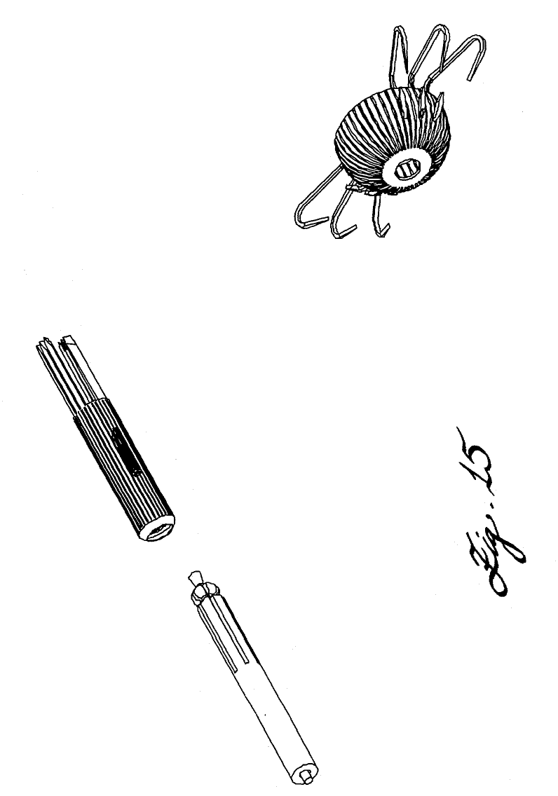

FIGS. 11a to 11c show the entire assembly of the insertion rod device 44, and illustrate how the stent basket 30 is loaded thereon. Referring to FIG. 11a, the stent basket 30 is positioned within the insertion rod device for delivery into the annular defect. The insertion rod device 44 further comprises a leg control knob 52, which is secured to the inner rod portion 46. The stent basket 30 is positioned over the inner rod portion 46, and advancement of the leg control knob 52 functions to release the stent retention legs 38. The stent retention legs 38 are in their unexpanded form prior to delivery. The insertion rod device 44 further comprises an outer tube 54 that is positioned over the inner rod portion 46 and the holding sleeve 48. The outer tube 54 is secured to a stent constraint knob 56. The stent constraint knob 56 is positioned between the outer tube 54 and a handle 58. Retracting the stent constraint knob 56 causes the stent basket 30 to expand radially.

Referring to FIG. 11b, the loading of the stent basket 30 onto the insertion rod device 44 is shown. The loading process uses a loading device 60, which changes the position of the stent basket 30 from the position shown in FIGS. 8a and 8b, to the position shown in FIGS. 11a and 11b. More specifically, in FIGS. 8a and 8b the reinforcement legs 38 are shown in an expanded position, whereas in FIGS. 11a and 11b the reinforcement legs are flattened to a compressed form where the legs are substantially linear. The loading device 60 is positioned over the insertion rod device and the stent basket and is engaged to compress the stent basket. By tightening a plurality of loading screws 62 on the loading fixture 60, the stent retention legs are deflected. At that point, retracting the inner rod 46 serves to capture the stent retention legs within grooves in the inner rod, and the loading screws are loosened. FIG. 11c illustrates the final steps for loading the stent basket onto the insertion rod device to prepare for delivery into the annular defect. More specifically, after the step of loosening the loading screws 62, the outer tube 54 and stent constraint knob 56 are positioned over the stent basket and into the loading fixture 62. The inner rod 46 is then retracted and holding sleeve 48 and stent basket 30 are positioned into outer tube 54. The stent basket 30 is then prepared for delivery into the annular defect by the insertion rod device.

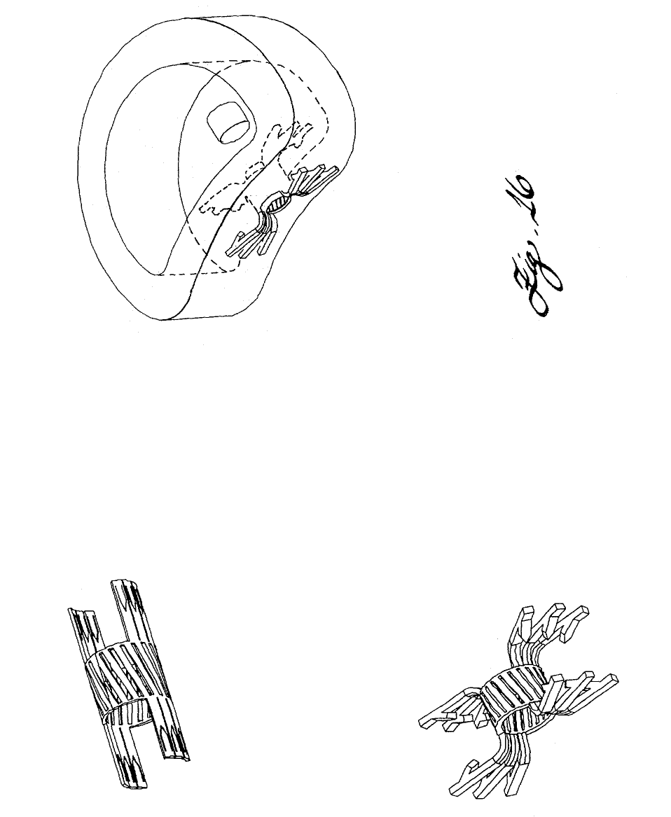

Referring to FIGS. 12 and 13, in conjunction with FIGS. 9 to 11, the delivery/insertion of the stent basket 30 into the annular defect 15 comprises the steps of first positioning the insertion rod device 44 into the annular defect 15. Next, the outer tube 54 is retracted such that the stent basket 30 expands radially. Next, referring to FIG. 13, the inner rod 46 is retracted, which assures that the stent retention legs 38 are deployed. At this point, the stent basket is positioned within the annular defect 15 and is engaged within the annulus. Next, the suture material 50 is severed, which releases the retaining arms 42 from the holding sleeve 48. The insertion rod device 44 is then removed from the patient's body and the stent basket is fully inserted into the annular defect.

The stent basket 30 provides repair to the annular defect by filling the empty space and by providing strength to the damaged portion of the annulus. Further, the stent basket prevents the nucleus from rupturing through the annulus and prevents collapse and damage to the annulus and disc.

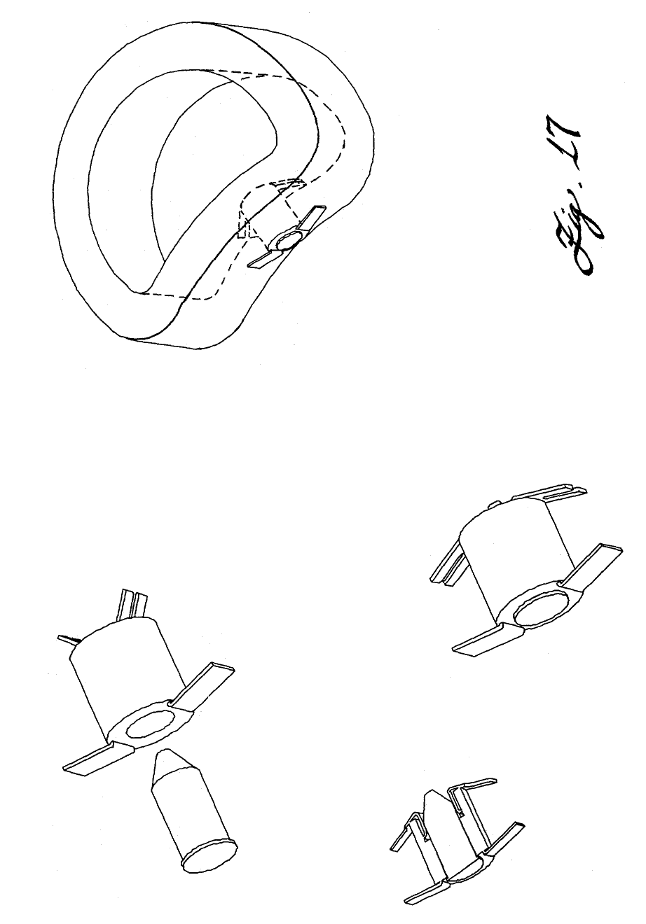

In addition to specific embodiments discussed above in detail, there are several other possible configurations for the present implant device. Below is a brief description of additional sample embodiments of implant devices of this invention that can be used for the repair of annular defects. Specifically, an additional thirteen configurations are shown in FIGS. 14 to 26. The same general concepts and principles discussed above are equally applicable to the embodiments shown in FIGS. 14 to 26. Accordingly, these embodiments will only be described generally with reference to the drawings, which in conjunction with the above-provided description provide sufficient disclosure to enable one of ordinary skill in the art to benefit and practice each of the embodiments without undue experimentation.

FIG. 14 shows another embodiment of the present invention, particularly a stent basket wherein a stent-like structure is delivered in a compressed state. A fibroelastic plug may or may not be inserted into the opening in the stent basket. Upon expansion, the hole in the annulus is filled and the locking legs lay against the inside wall. Barbs penetrate part way into the annulus and secure the device from dislodging into the nucleus. There are additional barbs from the mid-portion of the stent basket that go in the opposite direction to prevent the stent basket from going into the center of the nucleus. The basket may or may not have an opening that would provide a scaffold or for fibroblastic tissue repair.

It is understood that the implants of this invention are designed to accommodate changes that occur in the intervertebral discs to which they are inserted. An intervertebral disc, by its nature, undergoes expansion and contraction as a person moves in certain positions. The implants are designed to help a damaged disc having one or more of the implants inserted therein perform its original function. For example, if a patient's annular defect and/or nucleus enlarges when moving in a specific position, then the implant(s) would also expand to retain the contact of the implant(s) with the annular defect and/or nucleus, and thus mimic the annulus and/or nucleus. Similarly, if the annular defect and/or nucleus contracts, the implant(s) will contract to respond in the same manner as the residual annulus and/or nucleus. It is also understood that more than one implant can be used in a single intervertebral disc (i.e. a separate implant for the annular defect and nucleus).

With the stent basket of FIG. 14, as well as other embodiments of the present implant device, a T-handle inserter can be used for inserting the implant device. A tube (or sleeve) would fit over the implant. Once the stent basket was inserted into the annular defect, the tube (or sleeve) would be pulled back. As the threaded connection is still present, the device and sleeve now expands and the surgeon can gently pull back and rest the expanded device with barbs (optional) into the annulus. Next, the T-handle is unscrewed and then a tube would be inserted through the stent basket (optional) and the uncoiled portion delivered to fill the annular defect.

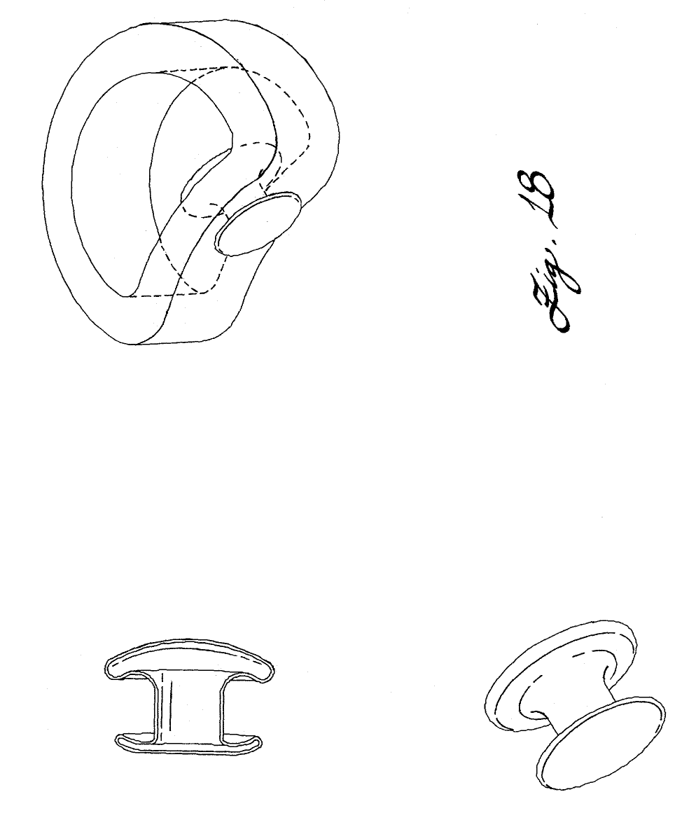

FIG. 15 shows another embodiment of the present invention, particularly an alternative stent basket which is similar to the stent basket in FIG. 14, however, it has a more flexible appearance, has thinner legs and barbs, and the barbs on the OD of the basket provide further fixation.

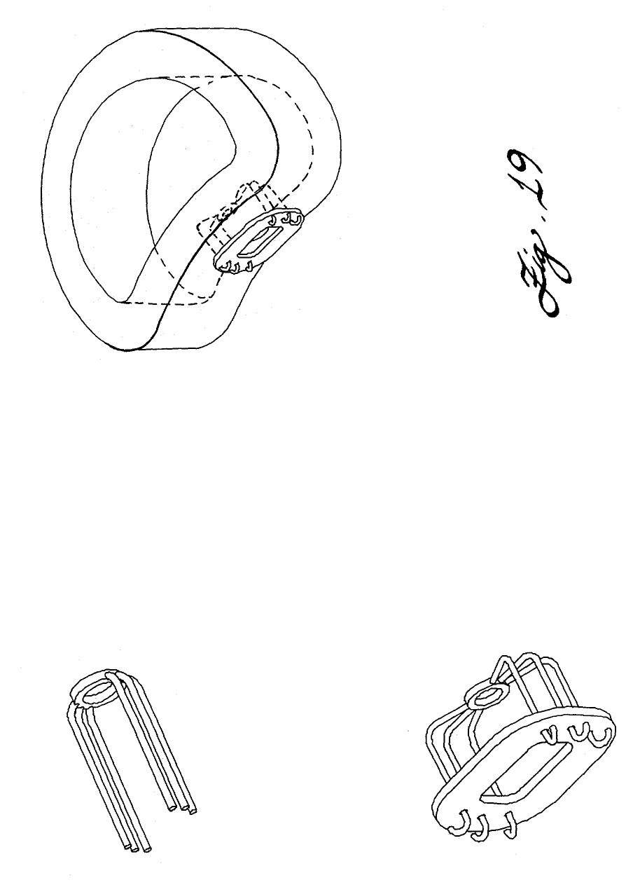

FIG. 16 shows another embodiment of the present invention, particularly a stent plug wherein a stent-like structure is delivered in a compressed state. Upon expansion, the hole in the annulus is filled and the locking legs lay against the inside and outside walls. Barbs may be provided to penetrate part way into the annulus and secure the opening from further expansion.

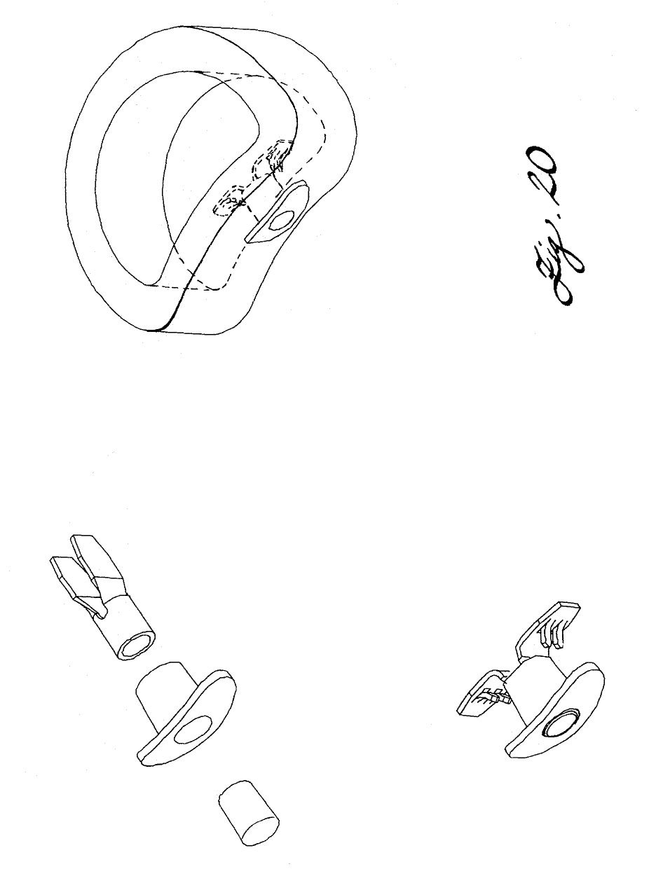

FIG. 17 shows another embodiment of the present invention, particularly a winged plug wherein a plug has rigid wings on the outside and moveable wings on the inside. The internal wings are locked in position by a sliding insert. When in position, the wings are locked by insertion of the pin. Sutures or barbs on the wings could further secure the device and the annulus opening.

FIG. 18 shows another embodiment of the present invention, particularly an inflatable plug wherein the plug is molded from an elastomer. For delivery, it is rolled or folded and pushed through the opening. After it is in place, the plug is filled with a liquid or gel through a valve (not shown). The geometry of the contact edges provides a large sealing area.

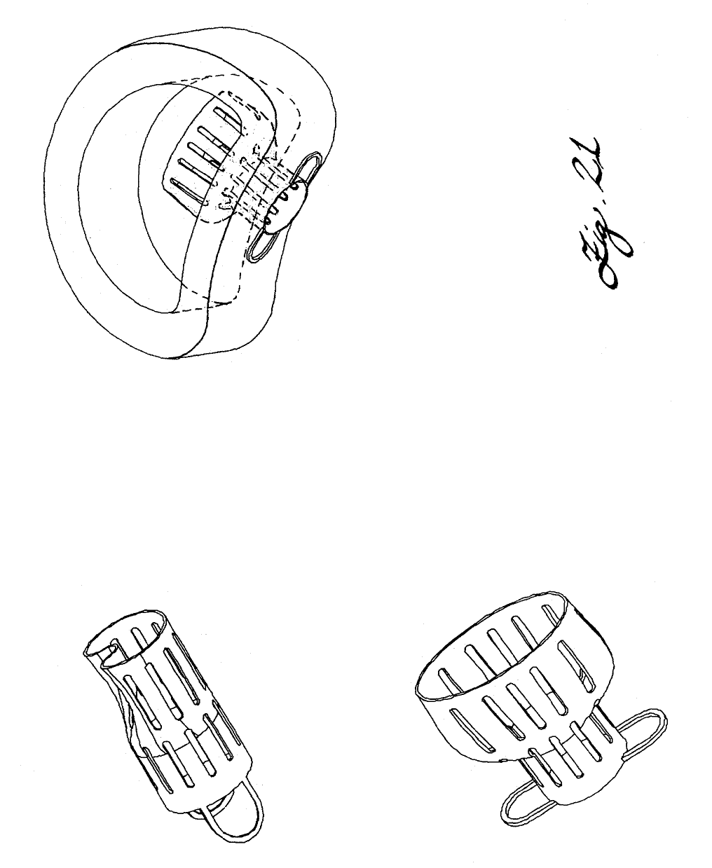

FIG. 19 shows another embodiment of the present invention, particularly a spider staple wherein a one piece staple is crimped or folded for delivery, expanded, then pulled outward through the annulus. A plate is installed to provide staple and plug (not shown) support. The staple is either crimped over or its shape set to provide a lock to the plate.

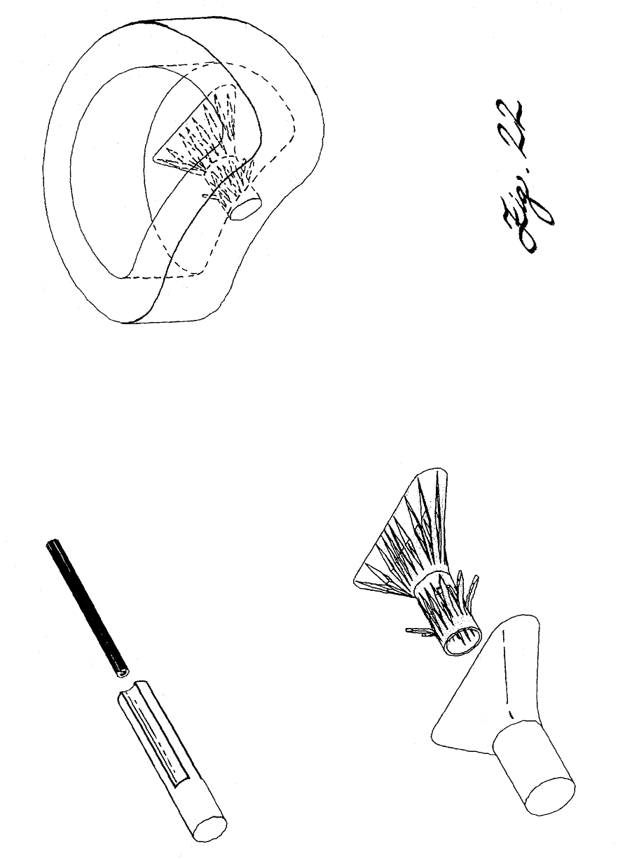

FIG. 20 shows another embodiment of the present invention, particularly a ratchet plug wherein an interior flange is shape set in an open position. Upon delivery it opens and seats against the inner annulus. A plate is inserted. The interface between the two parts is a ratchet which locks the parts in position and secures the two sides of the annulus under pressure. A plug is installed to seal the cavity.

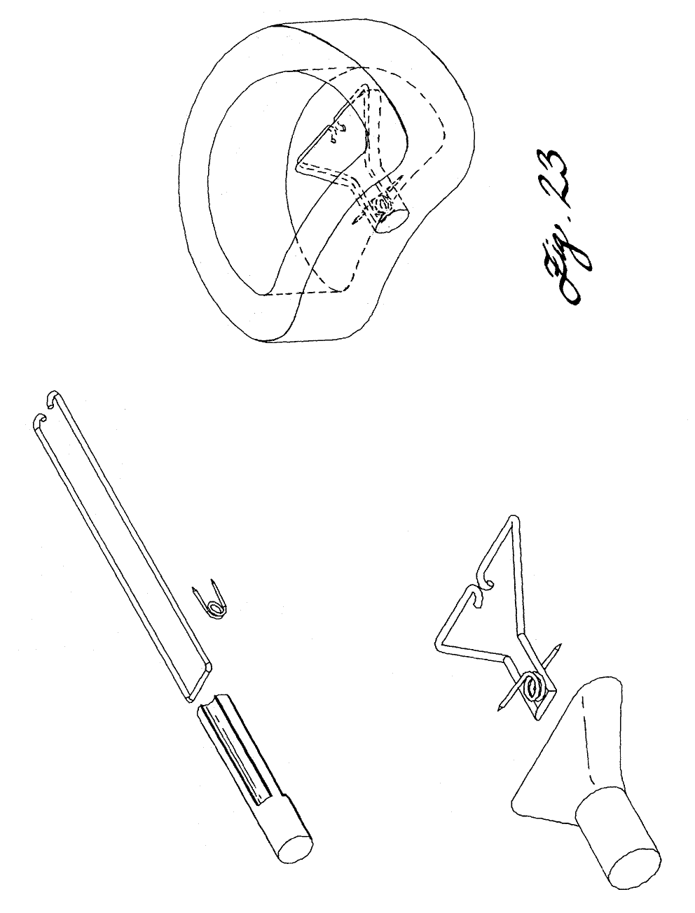

FIG. 21 shows another embodiment of the present invention, particularly a goblet plug wherein a stent-like structure with a fibrous plug (not shown) is delivered in a compressed state. Upon expansion, the hole in the annulus is filled and the plug is locked in place.

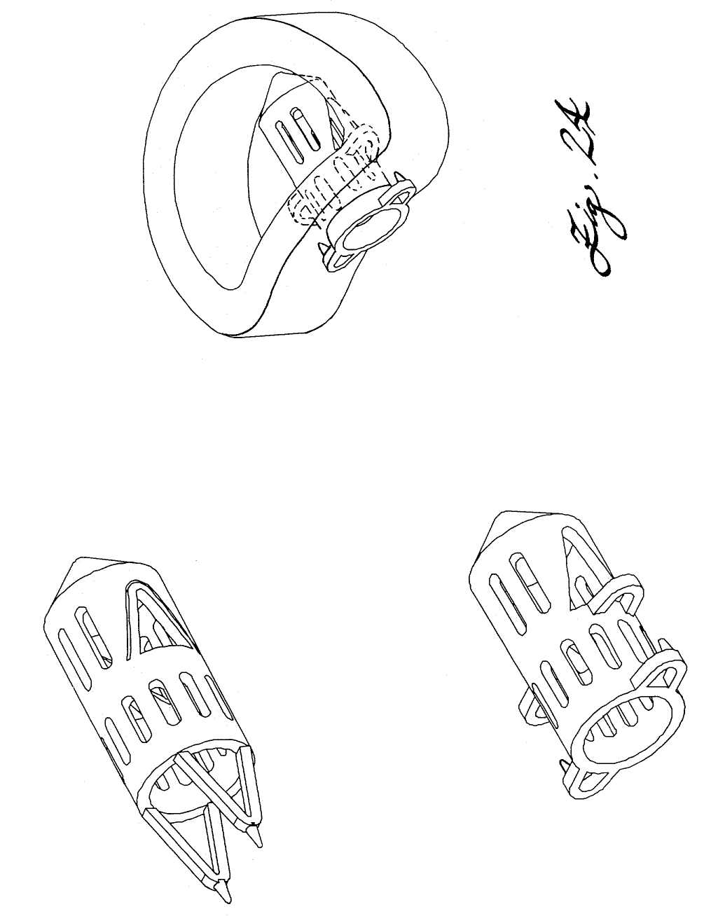

FIG. 22 shows another embodiment of the present invention, particularly an improved goblet device wherein a porous material for tissue growth is wrapped around an inverted wedge. The stent-like structure is delivered in a crimped state. Upon expansion, the stent is locked in place.

FIG. 23 shows another embodiment of the present invention, particularly another improved wire goblet device wherein porous material for tissue growth is wrapped around a wire frame. Upon expansion, the stent is locked in place with an independent barbed spring.

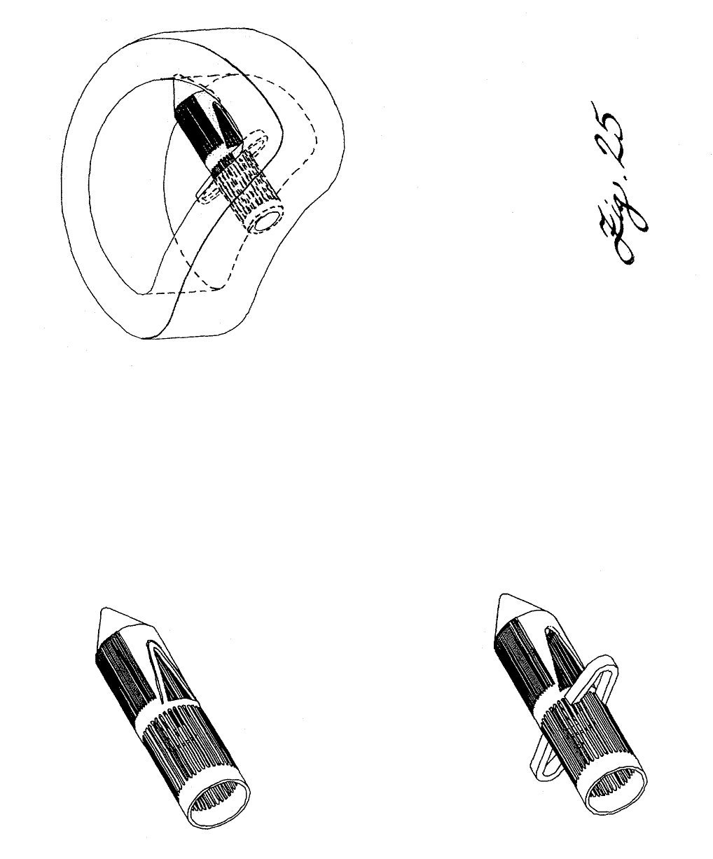

FIG. 24 shows another embodiment of the present invention, particularly a tubular plug wherein a stent-like structure with a fibrous plug (not shown) is delivered in a compressed state. Upon expansion, the hole in the annulus is filled and the locking legs lay against the inside and outside walls. Barbs may be provided to penetrate part way into the annulus and secure the opening from further expansion.

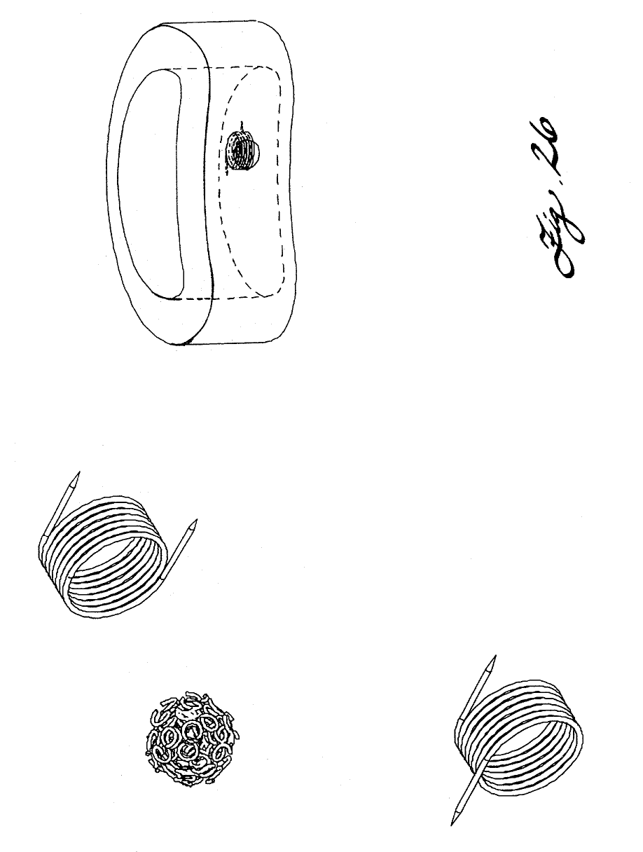

FIG. 25 shows another embodiment of the present invention, particularly an improved tubular plug wherein a stent-like structure is delivered in a compressed state. Upon expansion, the hole in the annulus is filled and the locking legs lay against the inside walls. A distal end may lay against the inside wall of the annulus to avoid further delivery.

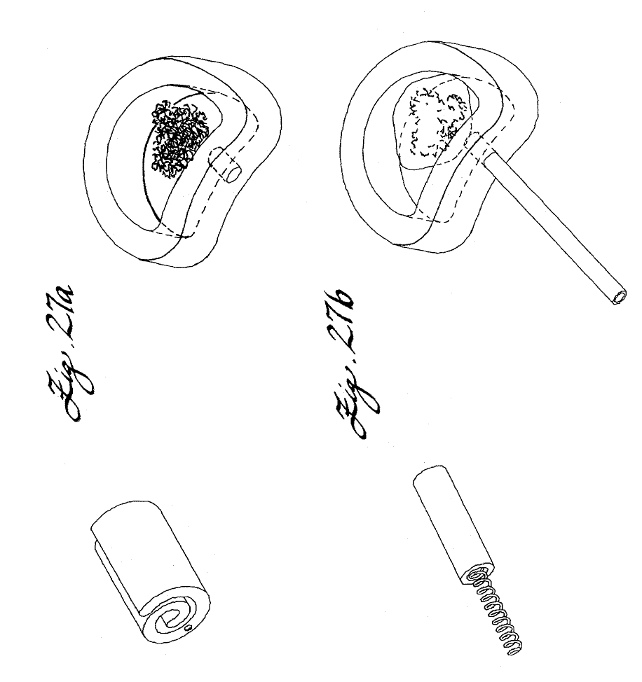

FIG. 26 shows another embodiment of the present invention, particularly a spring barb device wherein a simple spring structure is used and upon delivery, the barbs penetrate and lock the device in position. The structure is flexible and provides a scaffold for tissue growth. A filler of similar material or porous fiber could provide further scaffolding. Additionally, barb geometry could be altered to stop the opening from further expansion.

Repair and Restoration of the Nucleus

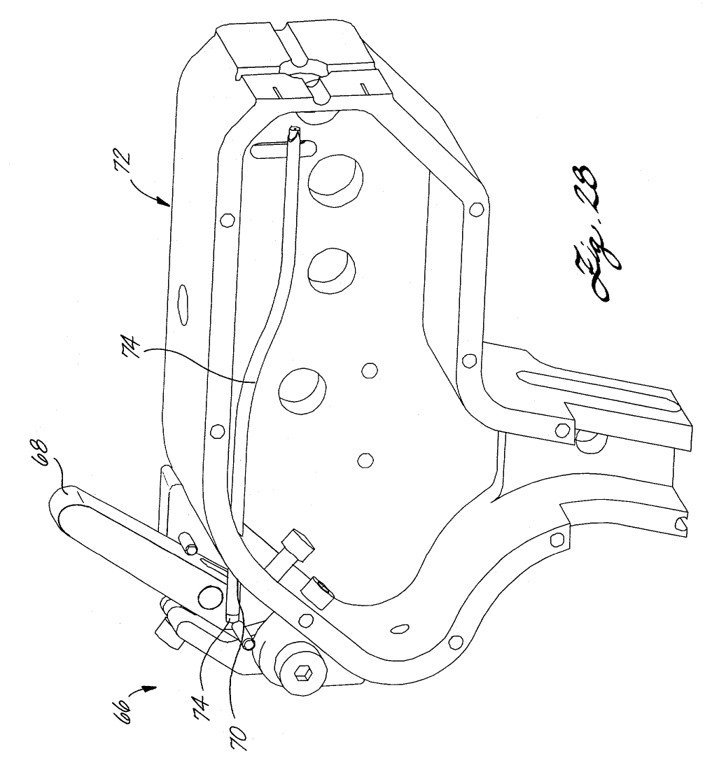

The present invention can also be used to repair and restore the nucleus portion of the disc. Generally, the teachings and disclosures provided above with respect to treatment of annular defects are applicable to the treatment and repair of the nucleus, and accordingly, will not be recited again. It is understood that the implants discussed above can be inserted into the nucleus to restore the nucleus. An additional implant that can be used to repair the nucleus is an SMA material that is inserted into the nucleus having a wire construction, and upon expansion, fills the entire nucleus area. Referring to FIG. 27a, a spring pad 64 is shown inserted into the nucleus 12. The spring pad 64 serves as a nucleus augmentation restoring flexibility, elasticity and height to the vertebral disc. The spring pad 64 comprises nitinol SMA, or other suitable flexible material, that was inserted into the nucleus in wire or small coil form. Enough material is deployed to fill the entire nucleus. The method of inserting the SMA wire or coil to form the spring pad 64 can be varied.

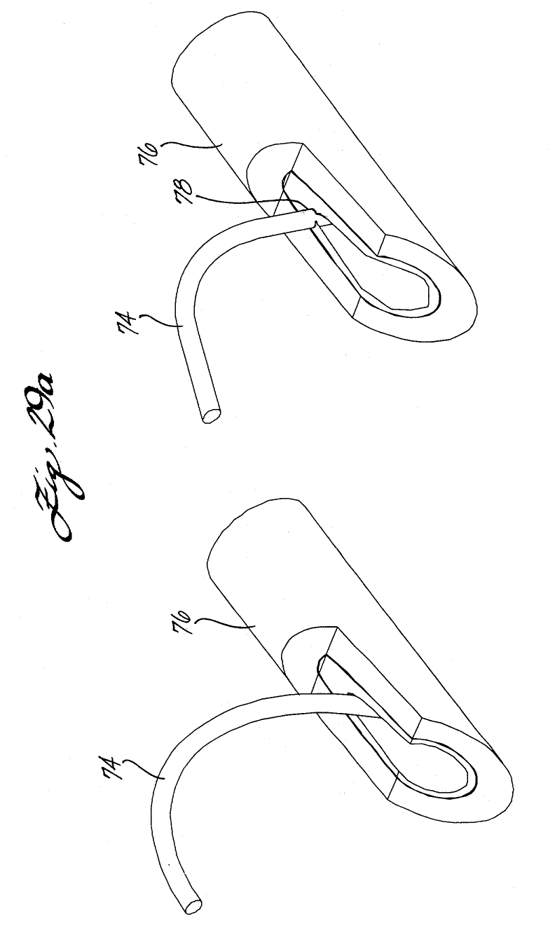

One method of delivering the implant into the nucleus includes use of an insertion device or delivery gun that transforms the coiled wire of the SMA to a straight wire as it passes through the delivery gun. Referring to FIG. 28, a delivery gun 66 is partially shown. The delivery gun comprises a retractable lever 68 that is manually positioned to allow access to an opening 70 that provides a controlled path through a chamber 72. A nitinol wire 74 is shown disposed through the opening 70 and positioned within the chamber 72, such that the retractable lever enables a user to feed the nitinol wire through the delivery gun and into the nucleus.

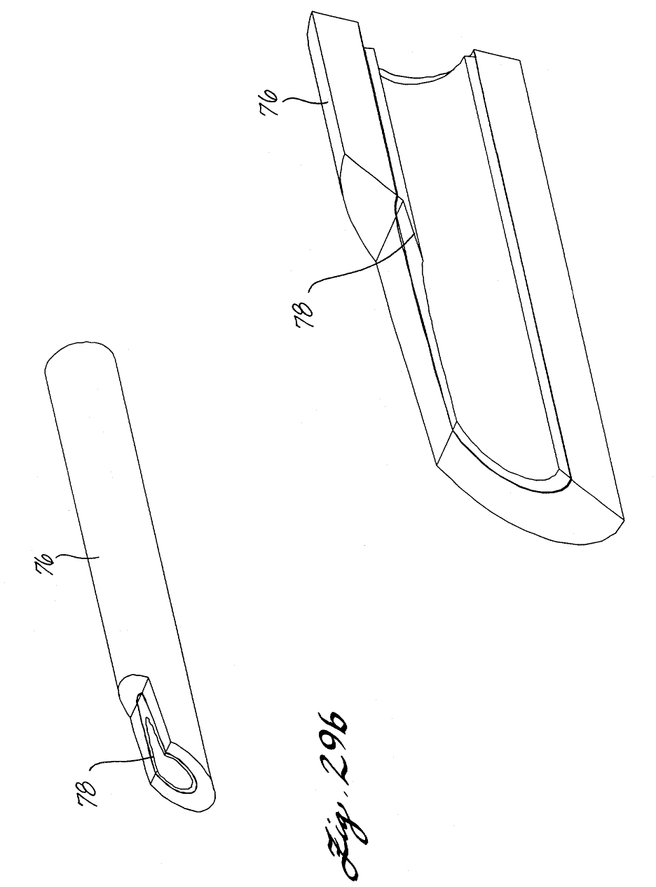

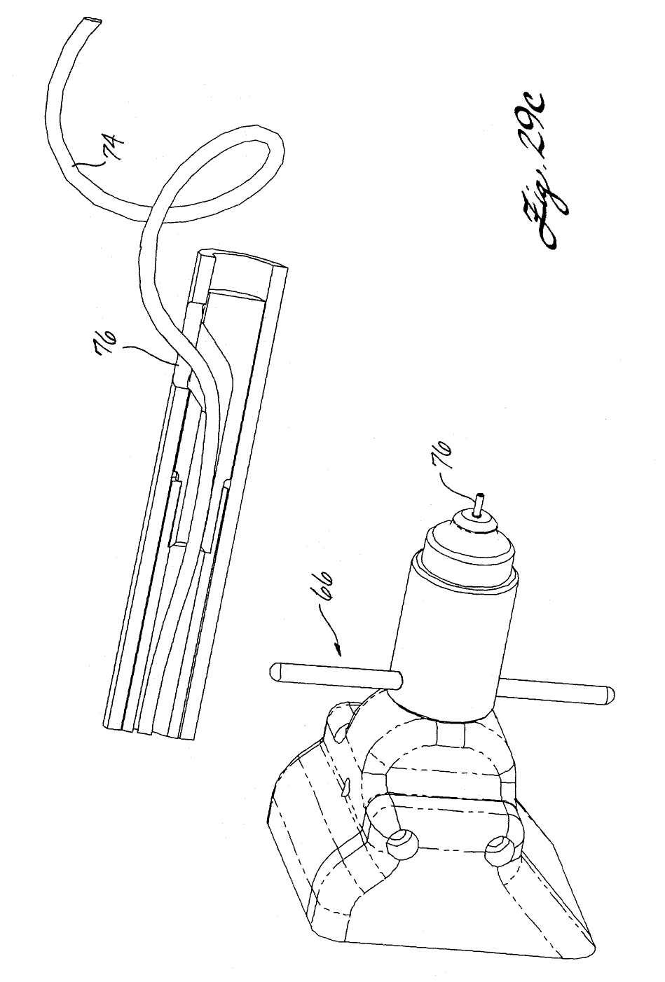

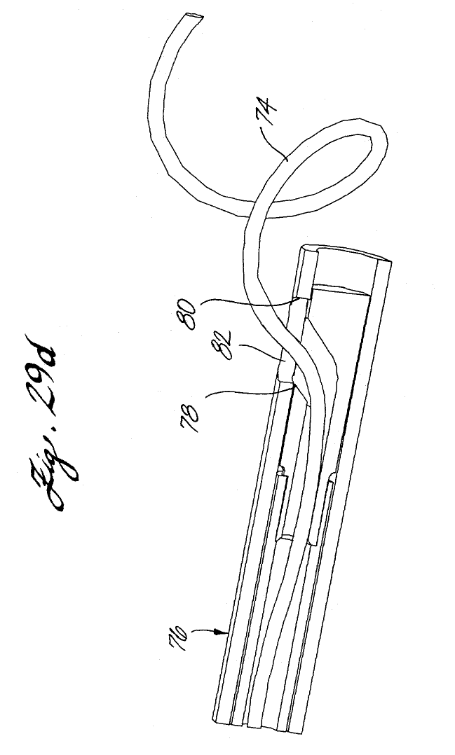

Referring to FIGS. 29a to 29d, there is a needle or cannula 76 positioned at an end of the delivery gun 66 that is positioned opposite the retractable lever 68 (shown in FIG. 28). Two types of needles are shown, namely (1) an end port needle shown in FIGS. 29a and 29b where, a notch is located at the top or bottom of the needle, and (2) a side port needle shown in FIGS. 29c and 29d where the notch is located at the side of the needle. Both types of needles share the same general construction and are referred to as the needle 76. The needle 76 is adapted for insertion into the nucleus and allows the nitinol wire 74 to pass therethrough. All of the needles may or may not be Teflon lined.

As shown in FIGS. 29a to 29d, the needle 76 includes a cutting edge or blade 78 that severs the nitinol wire 74 after the desired amount of nitinol wire has been inserted into the nucleus. The nitinol wire feeds smoothly through the needle into the nucleus until the direction is reversed. As shown in FIGS. 29a and 29b, when the direction of the nitinol wire is reversed, the wire is drawn into the blade, wherein it is notched, then sheared by the pull force. The needle 76 can comprise an outer needle 80 having a cut out 82 that draws the nitinol wire 74 back into the cutting edge. Further, as shown in FIGS. 29c and 29d, wire may be cut by a side cutting guillotine type cutter. In such a configuration, the wire shape memory alloy exits from a side port at the end of the needle. This will require special beveling of the needle within the cavity of the needle to allow the wire, or whatever the device shape is, to exit properly.

Additionally, the end of the shape memory wire or cable may or may not have a closed loop at each end. The advantage of having a closed loop, if present, is that no sharp ends are available for potential penetration into annular tissue and potential migration from the nucleus center into the edge of annulus. The implant may be configured such that closed loops form at the ends of the wire after expansion or transition of the implant.

The delivery gun transforms the coiled wire of the shape memory device to a straight wire as it passes through the delivery gun and needle to exit from the tip of the needle into the center of the nucleus. There, the wire recoils into the predetermined shape. The implant may go into the nucleus randomly or in a certain pattern (reproducible). Moreover, the nuclear restoring implant may go into a nucleus that has not been removed or, alternatively, some nucleus may require removal to create a small cavity for the implant.

Additionally, the delivery gun used to insert the wire may or may not have a replaceable cartridge filled with the preset coiled wire or pre-shaped memory implant, and may be powered or manual. Also, the wire can be loaded into the delivery gun and then cut to length by the gun, or can be first cut to length then loaded into the delivery gun.

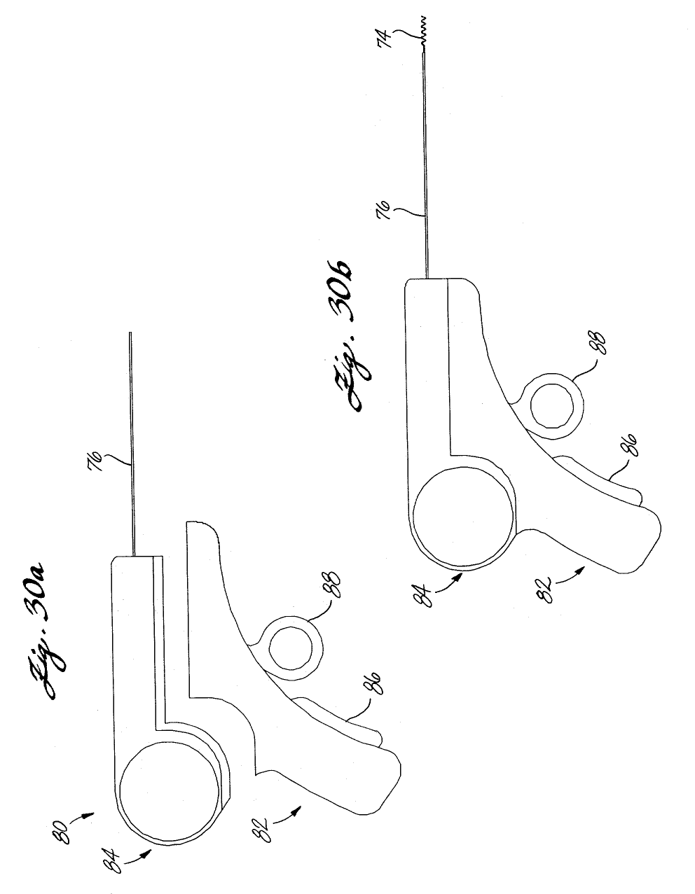

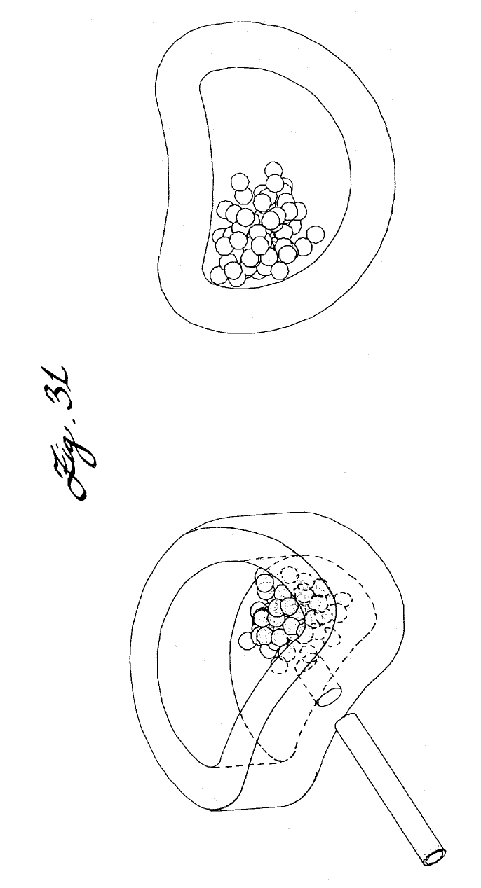

Another embodiment of a suitable delivery gun is shown in FIGS. 30a and 30b. Any of the features discussed above with respect to the delivery gun can be incorporated into this delivery gun as well, and some of the same reference numerals will be used to indicate similar components. FIG. 30a shows a delivery gun 80 having two separate portions that attach to form the single delivery gun 80 shown in FIG. 30b. The delivery gun 80 comprises a body 82 and a replaceable cartridge 84 that attaches to the body. The replaceable cartridge 84 is a housing for the nitinol wire 74, or any other suitable implant material being used for nuclear repair. Further, the replaceable cartridge mounts to the body to allow the user of the delivery gun to insert the needle 76 into the nuclear and then deliver the nitinol wire 74 through the needle into the nucleus.

With the delivery gun 80, the user controls the insertion and delivery of the nitinol wire by activating a trigger 86 and a clasp 88. The trigger 86 is compressed by the user to cause the nitinol wire to be dispensed through the cartridge 84 and needle 76 and into the nucleus. The clasp 88 is compressed to sever the nitinol wire at the needle tip. The structure of the needle cutting edge can be similar to those discussed above. When the cartridge 84 runs out of implant material, a new cartridge can be attached to the body of the delivery gun.

As shown in FIG. 27b, the wire or cable may or not be deployed into a bag or container made of Gore-Tex, polypropylene or some other material to contain it into the nucleus. The bag can be inserted into the nucleus by an suitable delivery device, and then the flexible bag is filled with a wire, coil, or other suitable material for expanding the nucleus.

FIG. 31 shows another embodiment of the present invention, particularly microcellular spheres wherein a microcellular elastomer is filled with gas bubbles. This allows for compressibility. The spherical shape allows for movement and self equalization of the filler. This concept could be for partial or complete nucleus replacement.

Treatment of Cancellous Bone Fractures

The present invention also can be used in different areas of the human body, including areas of cancellous bone fractures. These occur in multiple areas of the body including the distal radius, the plateau of the tibia adjacent to the knee joint, which generally results in collapse and distortion of the joint space or cancellous fracture of the heel. Other fractures amenable to the present implants include fractures in the thoracic or lumbar spine. The present implants can be inserted into such fractures and expand to fill the defect and reconstruct alignment.

The implant can be an SMA requiring activation (i.e. temperature or electrical) or can be a superelastic SMA or other suitable material. The implant is compressed into a very small volume for delivery into the fracture void, either directly or by cannula percutaneously, and then expands to fill the void. Just as with the implants for annular defects and nuclear repair, the implants for treatment of bone fractures can be made to any necessary shape and/or size.

Simple bone graft added to these sites for more successful healing would also be appropriate, either autogenous (from the patient) or cadaveric (from bone bank). Bone cement, such as methyl methacrylate or other synthetic polymers, can also be used.

As a result of the present implants, the common collapse seen in the healing process due to the soft spongy bone not having structural integrity can be avoided. Thus, significant shortening of the fracture and change of alignment of the joint and of the fracture can be avoided, and more successful healing results. This includes a better reduction of the fracture and better maintenance of the reduction as the fracture heals. Thus, the present implants successfully overcome the problems associated with known treatments for such fractures.

Each of the implants described with respect to annular repair, nuclear repair, and fracture repair may or may not be coated with titanium oxide or some other coating, potentially hydrophilic, to reduce wear debris. In fact, the implant may actually be coated with one or both of these coatings in order to reduce the likelihood of wear debris.

In addition to the specific features and embodiments described above, it is understood that the present invention includes all equivalents to the structures and features described herein, and is not to be limited to the disclosed embodiments. For example, the size, shape, and materials used to construct each of the implants can be varied depending on the specific application, as can the methods and devices used to insert them into the patient. Additionally, individuals skilled in the art to which the present expandable implants pertain will understand that variations and modifications to the embodiments described can be used beneficially without departing from the scope of the invention.

1. An expandable implant for intervertebral disc repair comprising:
a compressed form having a size adapted for insertion into a defect in the intervertebral disc;
a composition that allows the implant to expand from the compressed form into an expanded form after the implant is inserted into the defect; and
the expanded form having a configuration that fills the defect in the disc.

2. The expandable implant of claim 1 wherein the composition of the expandable implant comprises a shape memory alloy, wherein the expandable implant restores flexibility and provides support to residual intervertebral disc structure, and does not result in a fusion of intervertebral disc segments.

3. The expandable implant of claim 1 wherein the defect is an annular defect in an annular portion of the disc.

4. The expandable implant of claim 2 wherein the compressed form is a non-memory shape that is retained until the implant is activated by temperature or electrical current, such that activation transforms the expandable implant to a predetermined memory shape that defines the expanded form.

5. The expandable implant of claim 4 wherein the shape memory alloy is nitinol.

6. The expandable implant of claim 3 wherein the composition of the expandable implant is a superelastic shape memory alloy that changes from the compressed form to the expanded form automatically after the expandable implant is inserted into the annular defect.

7. The expandable implant of claim 1 wherein the defect is a nucleus of the disc, wherein a portion of the nucleus has ruptured through an annulus of the disc and has been surgically removed.

8. The expandable implant of claim 7 wherein the expandable implant is inserted into the nucleus in the compressed state and after the expandable implant has expanded to the expanded form the defect in the nucleus is filled.

9. The expandable implant of claim 8 wherein the composition of the expandable implant comprises a shape memory alloy.

10. The expandable implant of claim 9 wherein the compressed form is a non-memory shape that is retained until the implant is activated by temperature or electrical current, such that activation transforms the expandable implant to a predetermined memory shape that defines the expanded form.

11. The expandable implant of claim 10 wherein the shape memory alloy is nitinol.

12. The expandable implant of claim 8 wherein the composition of the expandable implant is a superelastic shape memory alloy that changes from the compressed form to the expanded form automatically after the expandable implant is inserted into the annular defect.

13. The expandable implant of claim 7 wherein the expandable implant is a shape memory alloy that enters the nucleus in the compressed form having a straight wire construction, and after insertion of the expandable implant is complete the straight wire construction transforms to a coil construction that defines the expanded form.

14. An expandable implant for treatment of an annular defect in an intervertebral disc comprising:
a body adapted for insertion into the annular defect;
the body adapted to radially expand to fill the annular defect; and
means for retaining the body within the annular defect.

15. The expandable implant of claim 14 wherein the means for retaining the expandable implant is selected from the group consisting of retention legs, barbs, and retention legs and barbs together.

16. The expandable implant of claim 15 wherein the retention legs and barbs are each adapted to at least partially penetrate annular tissue that defines the annular defect, such that the expandable implant is prevented from migration from its inserted location.

17. The expandable implant of claim 14 wherein the expandable implant is made of a shape memory alloy, wherein the expandable implant restores flexibility and provides support to residual annulus structure, and does not result in a fusion of intervertebral disc segments.

18. The expandable implant of claim 17 wherein the body is inserted into the annular defect in a compressed, non-memory shape.

19. The expandable implant of claim 18 wherein the compressed, non-memory shape transforms to an expanded, predetermined memory shape after the expandable implant has been inserted into the annular defect.

20. The expandable implant of claim 14 wherein the expandable implant is made of a superelastic shape memory alloy that changes from a compressed form to an expanded form automatically after the expandable implant is inserted into the annular defect.

21. An expandable implant for nuclear repair of an intervertebral disc comprising:
a pre-insertion shape adapted for insertion into a nucleus of the intervertebral disc;
a composition that allows the pre-insertion shape to be transformed to a post-insertion shape after the expandable implant is inserted into the nucleus; and
the post-insertion shape defining a larger volume than the pre-insertion shape, such that the expandable implant fills the nucleus.

22. The expandable implant of claim 21 wherein the composition comprises a shape memory alloy, wherein the expandable implant restores flexibility and provides support to residual nucleus structure, and does not result in a fusion of intervertebral disc segments.

23. The expandable implant of claim 22 wherein the expandable implant is inserted by a delivery device into the nucleus.

24. The expandable implant of claim 23 wherein the delivery device comprises a needle adapted to transport the expandable implant into the nucleus.

25. The expandable implant of claim 24 wherein the expandable implant is a nitinol wire that passes through the needle in a non-coiled shape.

26. The expandable implant of claim 25 wherein the delivery device further comprises means for controlling the amount of nitinol wire passing through the needle into the nucleus and for cutting the nitinol wire to separate the nitinol wire from the delivery device.

27. The expandable implant of claim 26 wherein the nitinol wire inserted within the nucleus transforms to a coiled shape that defines the post-insertion shape of the expandable implant.

28. The expandable implant of claim 27 wherein the expandable implant restores the height and elasticity of the nucleus.

29. A shape memory alloy implant for treatment of cancellous bone fractures comprising:
a compressed form adapted for insertion into areas of cancellous bone fractures; and
an expanded form that results from insertion of the compressed form into the cancellous bone fracture, wherein the expanded form fills in the cancellous bone fracture.

30. The shape memory alloy implant of claim 29 wherein the cancellous bone fractures comprises distal radius fractures, tibial plateau fractures, and calcaneous fractures.

31. A delivery device for inserting an implant into an intervertebral disc comprising:
means for retaining the implant within the device while the device is positioned into the intervertebral disc; and
means for controllably releasing the implant into the intervertebral disc.

32. The delivery device of claim 31 being adapted for inserting the implant into an annular defect in the intervertebral disc, and the means for retaining the implant comprises an inner rod to which the implant has been adjoined and an outer rod that is positioned over the inner rod until the implant is released.

33. The delivery device of claim 32 wherein the implant is retained in a compressed form by the inner rod until the implant is release from the device, at which point the implant transforms to an expanded form.

34. The delivery device of claim 32 wherein the means for controllably releasing the implant comprises one or more knobs that retract the inner rod and outer rod.

35. The delivery device of claim 31 being adapted for inserting an implant into a nucleus of the intervertebral disc, and the means for retaining the implant comprises a chamber and a needle, wherein the implant is passed through the chamber into the needle and into the intervertebral disc.

36. The delivery device of claim 36 wherein the chamber retains a predetermined amount of the implant.

37. The delivery device of claim 36 wherein the means for controllably releasing the implant comprises an activation trigger that feeds the implant through the chamber and needle.

38. The delivery device of claim 37 wherein the means for controllably releasing the implant further comprises a clasp that activates a cutting edge within the needle that severs the implant being fed therethrough.

39. The delivery device of claim 38 wherein the means for retaining the implant and the means for controllably releasing the implant are positioned into two separate portions of the delivery device that are designed to cooperatively engage.

40. A method of inserting an implant into an intervertebral disc comprising:
loading the implant into a delivery device adapted for insertion into the intervertebral disc, wherein the implant is in a compressed form;
inserting the delivery device into the intervertebral disc; and
releasing the implant from the delivery device into the intervertebral disc, wherein the implant transforms from the compressed form to an expanded form designed to repair the intervertebral disc.
